(19) United States
(12) Patent Application Publication
(10) Pub. No.: US 2022/0218748 A1
(43) Pub. Date: Jul. 14, 2022

(54) CELLS FOR IMPROVED IMMUNOTHERAPY AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: David A. Scheinberg, New York, NY (US); Leila Peraro, Cambridge, MA (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 17/590,585

(22) Filed: Feb. 1, 2022

Related U.S. Application Data
(63) Continuation of application No. PCT/US2020/044159, filed on Jul. 30, 2020.
(60) Provisional application No. 62/881,467, filed on Aug. 1, 2019.

(51) Int. Cl. A61K 35/17; C07K 14/725; C07K 16/28; C12N 9/64

(52) U.S. Cl. CPC A61K 35/17; C07K 14/7051; C07K 16/2833; C12N 9/6472; C07K 2319/03; C07K 2317/24; C07K 2317/622; C07K 2317/734; C12Y 304/22

(57) ABSTRACT

The presently disclosed subject matter provides cells and compositions for improved immunotherapy and methods of using such cells and compositions. It relates to cells comprising a ligand-recognizing receptor (e.g., an antigen-recognizing receptor, e.g., a chimeric antigen receptor (CAR) or a T-cell Receptor (TCR)) and an IgG-degrading enzyme or a fragment thereof. The IgG-degrading enzyme rapidly cleaves IgG. The IgG-degrading enzyme serves as a biomolecular shield against the host humoral response. The cells have increased resistance to host humoral response (e.g., an antibody-driven host humoral response), which allows for prolonged persistence of the cells, leading to enhanced activity of the cells.

### CROSS-REFERENCE TO RELATED APPLICATIONS

This application is a Continuation of International Patent Application No. PCT/US2020/044159, filed Jul. 30, 2020, which claims priority to U.S. Provisional Patent Application Ser. No. 62/881,467, filed Aug. 1, 2019, the content of each of which is incorporated by reference in its entirety, and to each of which priority is claimed.

### GRANT INFORMATION

This invention was made with government support under Grant No. P30 CA008747 from National Cancer Institute of the National Institutes of Health. The government has certain rights in the invention.

### SEQUENCE LISTING

The specification further incorporates by reference the Sequence Listing submitted herewith via EFS on Feb. 1, 2022. Pursuant to 37 C.F.R. § 1.52(e)(5), the Sequence Listing text file, identified as 0893390211 SL.txt, is 52,754 bytes and was created on Feb. 1, 2022. The Sequence Listing electronically filed herewith, does not extend beyond the scope of the specification and thus does not contain new matter.

### 1. INTRODUCTION

The presently disclosed subject matter provides cells and compositions for improved immunotherapy and methods of using such cells and compositions. It relates to cells comprising a ligand-recognizing receptor (e.g., an antigen-recognizing receptor, e.g., a chimeric antigen receptor (CAR) or a T-cell Receptor (TCR)) and an IgG-degrading enzyme or a fragment thereof. The IgG-degrading enzyme rapidly cleaves IgG. The IgG-degrading enzyme serves as a biomolecular shield against the host humoral response. The cells have increased resistance to host humoral response (e.g., an antibody-driven host humoral response), which allows for prolonged persistence of the cells, leading to enhanced activity of the cells.

### 2. BACKGROUND OF THE INVENTION

Synthetic immunology and synthetic biology harness immune cells to kill tumor cells or to treat other important diseases. Areas of rapid growth in synthetic immunology and biology are in the use of adoptive cell transfer, stem cell transplant, organ transplants, CRISPR gene editing, genetic therapies, and CAR-T cell therapies. In any situation in which an altered or engineered cell is introduced into a subject, the host (the subject) may mount an immune response to the cell or tissue because it is foreign or contains foreign genes and proteins, that are not normally found in the host. The consequences of this immune recognition can be neutralization of the therapeutic effect, rejection of the tissue or cells, and/or failure of the therapeutic intent, etc. Therefore, there is a need for engineered cells having increased resistance to host humoral responses.

### 3. SUMMARY OF THE INVENTION

The presently disclosed subject matter provides cells comprising (a) a ligand-recognizing receptor, and (b) an IgG-degrading enzyme or a fragment thereof In certain embodiments, the IgG-degrading enzyme is secreted. In certain embodiments, the IgG-degrading enzyme is membrane bound. In certain embodiments, the cell further comprises (c) a transmembrane domain attached to the IgG-degrading enzyme. The transmembrane domain can be attached to the C-terminus of the IgG-degrading enzyme. In certain embodiments, the transmembrane domain attached to the IgG-degrading enzyme comprises a CD8 polypeptide.

In certain embodiments, the IgG-degrading enzyme is selected from IgG-degrading enzyme of S. pyogenes (IdeS), IgG-degrading enzyme of S. equi subsp. zooepidemicus (IdeZ), IgG-degrading enzyme of S. equi subsp. equi. (IdeE), an endoglycosidase from Streptococcus pyogenes (EndoS), and a streptococcal cysteine proteinase from Streptococcus pyogenes (SpeB).

In certain embodiments, the ligand-recognizing receptor is exogenous or endogenous. In certain embodiments, the ligand-recognizing receptor is recombinantly expressed. In certain embodiments, the ligand-recognizing receptor is expressed from a vector. In certain embodiments, the IgG-degrading enzyme is expressed from a vector.

In certain embodiments, the cell is a responsive cell. In certain embodiments, the cell is a responsive cell, e.g., an immunoresponsive cell. In certain embodiments, the cell is an activatable cell. In certain embodiments, the cell is selected from T cells, Natural Killer (NK) cells, B cells, macrophages, monocytes, dendritic cells, stem cells, normal tissue cells (e.g., from kidney, liver, lung, bone marrow, or pancreas) and combinations thereof. In certain embodiments, the cell is a T cell.

In certain embodiments, the ligand-recognizing receptor binds to an antigen. The antigen can be a tumor antigen, a pathogen antigen, a normal cell antigen, an HLA antigen or an alloantigen. In certain embodiments, the antigen is a normal cell antigen. In certain embodiments, the alloantigen is a minor histocompatibility alloantigen.

In certain embodiments, the antigen is a tumor antigen. In certain embodiments, the tumor antigen is CD19.

In certain embodiments, the ligand-recognizing receptor is a T cell receptor (TCR) or a chimeric antigen receptor (CAR). In certain embodiments, the ligand-recognizing receptor is a CAR. In certain embodiments, the CAR comprises an extracellular antigen-binding domain, a transmembrane domain, and an intracellular signaling domain. In certain embodiments, the extracellular antigen-binding domain of the CAR comprises a single chain variable fragment (scFv).

In certain embodiments, the intracellular signaling domain of the CAR comprises a CD3ζ polypeptide. In certain embodiments, the transmembrane domain comprises a CD8 polypeptide. In certain embodiments, the intracellular signaling domain of the CAR further comprises at least one co-stimulatory signaling domain. In certain embodiments, the at least one co-stimulatory domain comprises a CD28 polypeptide, a 4-1BB polypeptide, or a combination thereof In certain embodiments, wherein the at least one co-stimulatory domain comprises a 4-1BB polypeptide. In certain embodiments, the intracellular signaling domain of the CAR comprises two co-stimulatory signaling domains.

In certain embodiments, the IgG-degrading enzyme cleaves an IgG, thereby preventing an IgG antibody from killing the cell. In certain embodiments, the IgG-degrading enzyme cleaves an

IgG, thereby allowing the remaining fragment of the IgG to retain the binding to the cell, which protects the cell from one or more cytotoxic antibodies. In certain embodiments, the one or more cytotoxic antibodies bind to the same site as the IgG and kill the cell. Therefore, the process creates a protective shield.

The presently disclosed subject matter also provides compositions comprising the cells described herein. In certain embodiments, the composition is a pharmaceutical composition further comprising a pharmaceutically acceptable excipient. In certain embodiments, the composition is for treating a neoplasia.

Furthermore, the presently disclosed subject matter provides methods for producing a cell disclosed herein. In certain embodiments, the method comprises introducing into a cell (a) a first polynucleotide encoding a ligand-recognizing receptor; and (b) a second polynucleotide encoding an IgG-degrading enzyme or a fragment thereof. In certain embodiments, (a) and/or (b) are introduced to the cell genetically. In certain embodiments, the first polynucleotide is optionally operably linked to a promoter element. In certain embodiments, the second polynucleotide is optionally operably linked to a promoter element. In certain embodiments, one or both of the first and second polynucleotides are comprised in a vector. In certain embodiments, the first and second polynucleotides are comprised in two different vectors. In certain embodiments, the vector is a retroviral vector. In certain embodiments, the vector is a lentiviral vector. In certain embodiments, the vector is encoded in a mRNA molecule.

The presently disclosed subject matter further provides nucleic acid compositions. In certain embodiments, the nucleic acid composition comprises (a) a first polynucleotide encoding a ligand-recognizing receptor and (b) a second polynucleotide encoding an IgG-degrading enzyme or a fragment thereof. In certain embodiments, the first polynucleotide is operably linked to a promoter element. In certain embodiments, the second polynucleotide is operably linked to a promoter element. In certain embodiments, one or both of the first and second polynucleotides are comprised in a vector. In certain embodiments, the first and second polynucleotides are comprised in two different vectors. In certain embodiments, the vector is a retroviral vector. In certain embodiments, the vector is a lentiviral vector. In certain embodiments, the vector is encoded in a mRNA molecule.

Also provided are vectors comprising the nucleic acid composition described herein.

The presently disclosed subject matter further provides kits comprising a cell described herein, a composition described herein, a nucleic acid composition described herein, or a vector described herein. In certain embodiments, the kit further comprises written instructions for treating and/or preventing a neoplasia, a pathogen infection, and/or an autoimmune disorder.

The presently disclosed subject matter also provides various methods. The presently disclosed subject matter provides methods of reducing tumor burden in a subject. In certain embodiments, the method comprises administering to the subject an effective amount of a cell described herein, a composition described herein, a nucleic acid composition described herein, or a vector described herein. In certain embodiments, the method reduces the number of tumor cells, reduces tumor size, and/or eradicates the tumor in the subject.

The presently disclosed subject matter provides methods of treating and/or preventing a neoplasia, a pathogen infection, and/or an autoimmune disorder. In certain embodiments, the method comprises administering to the subject an effective amount a cell described herein, a composition described herein, a nucleic acid composition described herein, or a vector described herein.

The presently disclosed subject matter provides methods of lengthening survival of a subject having a neoplasia, a pathogen infection, and/or an autoimmune disorder. In certain embodiments, the method comprises administering to the subject an effective amount of a cell described herein, a composition described herein, a nucleic acid composition described herein, or a vector described herein.

In certain embodiments, the neoplasia is selected from acute myeloid leukemia (AML), lymphoblastic leukemia (ALL), chronic lymphocytic leukemia (CLL), chronic myeloid leukemia (CIVIL), multiple myeloma, non-Hodgkin's lymphoma, Hodgkin's lymphoma breast cancer, ovarian cancer, mesothelioma, glioblastoma, colorectal cancer, and pancreas cancer.

The presently disclosed subject matter provides methods of reducing and/or preventing an antibody-mediated rejection of cells and/or tissues in a subject who receives an organ transplant. In certain embodiments, the transplant is an allogeneic transplant (allotransplant). In certain embodiments, the subject receives the cells, composition, or nucleic acid composition prior to the organ transplant.

The presently disclosed subject matter further provides methods of reducing and/or preventing an antibody-mediated rejection of cells and/or tissues in a subject who receives a cell therapy. In certain embodiments, the cell and/or tissues are autologous or allogeneic. In certain embodiments, the cell and/or tissues are used in the cell therapy.

In certain embodiments, the method comprises administering an effective amount of a cell described herein, a composition described herein, a nucleic acid composition described herein, or a vector described herein.

### 4. BRIEF DESCRIPTION OF THE FIGURES

FIGS. 1A and 1B depict vectors and cells in accordance with certain embodiments of the presently disclosed subject matter. FIG. 1A depicts vectors and cells in accordance with certain embodiments of the presently disclosed subject matter, e.g., the IgG degrading enzymes are on the cells. FIG. 1B depicts vectors and cells in accordance with certain embodiments of the presently disclosed subject matter, e.g., the IgG degrading enzymes are secreted from the cells.

FIG. 2 depicts IdeS expression in mammalian cells. HEK293t cells were transiently transfected with the membrane-bound (IdeS-tm) and secreted (IdeS-sec) versions of IdeS. The cell lysates and supernatant fluid were analyzed by western immunoblot using an anti-HA antibody and the IdeS protein was detected in the cell or secreted fluid, respectively. Un-transduced HEK293t cells showed no IdeS protein. (Left lane in each gel is molecular weight marker.).

FIGS. 3A-3C depict cell-expressed IdeS cleavage activity over time. HEK 293t cells were transiently transfected with IdeS-tm. FIG. 3A shows 48 hr post transfection, where IgG was added to the wells of a 24-well plate, then removed at different time points and quenched using Laemmli buffer. The samples were analyzed by SDS-PAGE. FIG. 3B shows gel stained with Coomassie. Cleaved Ig appeared within 5 min and increased over time. FIG. 3C shows blot with an anti-human Fc-specific Antibody. Cleaved Ig as Fc fragment appeared within 5 min and increased over time.

FIGS. 4A and 4B depict cleavage by IdeS. An ELISA-based assay was used to analyze the extent of cleavage of IgG by IdeS. FIG. 4A shows a standard curve validation of the assay using recombinant IdeS. FIG. 4B shows confirmation of expression of IdeS in HEK293t cells by western immunoblot and cleavage activity assessed by ELISA at different time points.

FIGS. 5A-5B depict that that cell-secreted IdeS can cleave antibody bound on neighboring cells. Raji cells were incubated with Rituximab for 30 min. The Raji cells were then co-cultured overnight with HEK293t expressing the 19BBz construct alone, IdeS-tm 19BBz, or IdeS-sec 19BBz. The extent of cleavage was evaluated using a labeled anti-Fc secondary ab, and measured via flow cytometry. FIG. 5A represents histograms showing extent of anti-Fc fluorescence of Raji cells. FIG. 5B shows bar graph with Mean Fluorescence intensity (MFI) for each histogram.

FIG. 6 depicts killing of tumor cells by the cells of the presently disclosed subject matter. Untransduced T cells, T cells comprising 19BBz CAR and a membrane-bound IdeS, T cells comprising 19BBz CAR and secreting IdeS, and 19BBz CAR T-cells were incubated with Raji cells expressing Firefly luciferase for 18 hrs. Cell viability of Raji cells was assessed by adding luciferin and measuring bioluminescence. IdeS shielded CAR T cells were functionally as active as unshielded CAR T cells.

FIG. 7 depicts that the cells of the presently disclosed subject matter were protected against complement-dependent cytotoxicity (CDC). Untransduced T cells, T cells comprising 19BBz CAR and a membrane-bound IdeS, T cells comprising 19BBz CAR and secreting IdeS, andl9BBz CAR T-cells were treated with different concentrations of rabbit antithymocyte globulin (ATG) followed by the addition of rabbit serum and incubated for one hour. Cell viability was measured via Cell Titer Glo. T cells comprising 19BBz CAR and a membrane-bound IdeS and T cells comprising 19BBz CAR and secreting IdeS cleaved off the Fc fragments of IgG thus evading CDC and remained alive.

FIG. 8 depicts an exemplified mechanism of action of the cells of the presently disclosed subject matter. IgG antibodies bind to cell surface antigens and receptors leading to cell death via CDC, ADCC and opsonization. The cells of the presently disclosed subject matter express IdeS, an enzyme which cleaves IgG below the hinge region, releasing Fc fragments. The cells of the presently disclosed subject matter remain coated in F(ab′)₂ fragments, which prevent further antibody from binding.

FIG. 9 depicts the activity of CART cells expressing an IdeS. CART cells expressing IdeS cleaved IgG Fc and maintained F(ab′)₂ shield. CAR T cells were treated with 1 μg/mL of anti-thymocyte globulin (ATG) and incubated overnight. Cells were washed and analyzed with either anti-Fc (top) or anti-Fab (bottom) labelled antibodies. The median fluorescence intensity was plotted in bar graphs on the right.

FIG. 10 depicts that the presently disclosed cells were protected against antibody-dependent cellular cytotoxicity (ADCC). IdeS-tm 19BBz T-cells, IdeS-sec 19BBz T-cells, and 19BBz T-cells without IdeS were treated with different doses of anti-thymocyte globulin (ATG) and subsequently with human PBMCs. Both IdeS-tm 19BBz T-cells and IdeS-sec 19BBz T-cells were protected from lysis compared to the 19BBz T-cells without IdeS.

FIGS. 11A-11C depict that the presently disclosed cells can cleaved serum IgG from a kidney transplant patient and were protected from CDC. FIG. 11A depicts that serum derived from a kidney transplant patient (patient 2) containing anti-HLA antibodies causing rejection was shown to bind A02+cells by flow cytometry. FIG. 11B depicts that the serum from patient 2 was cleaved by A02+IdeS-tm 19BBz T-cells and IdeS-sec 19BBz T-cells, as verified by flow cytometry. FIG. 11C depicts that A02+IdeS-tm 19BBz T-cells and IdeS-sec 19BBz T-cells were also protected from complement killing (CDC) mediated by patient 2 serum.

FIG. 12 depicts that the presently disclosed cells cleaved human polyclonal IgG in vivo. Human T cells were transduced with the 19BBz without IdeS CAR (Lanes from left: #1-2) or IdeS-tm 19BBz CAR (transmembrane form) (Lanes from left: #3-5) and IdeS-sec 19BBz (secreted form)

(Lanes from left: #6-8). CAR T cells were injected i.p. in NSG mice and after 24 hr human polyclonal IgG was also injected i.p. Cleavage of IgG was assessed by performing an ip lavage using PBS, purifying the samples using magnetic protein G beads, and analyzing by Western Blot using an anti-human Fc-specific HRP secondary antibody. Un-cleaved heavy chain can be observed around 55 kDa (lane #9), while cleaved Fc fragments are present around 25 kDa (arrow).

### 5. DETAILED DESCRIPTION OF THE INVENTION

The following Detailed Description, given by way of example, but not intended to limit the presently disclosed subject matter to specific embodiments described, may be understood in conjunction with the accompanying drawings.

The presently disclosed subject matter provides cells, including genetically modified immunoresponsive cells (e.g., T cells or NK cells) comprising a ligand-recognizing receptor (e.g., a TCR or a CAR) and an IgG-degrading enzyme or a fragment thereof, and compositions comprising such cells. The presently disclosed subject matter also provides methods of producing such cells, and methods of using such cells and compositions comprising thereof. The presently disclosed subject matter also provides use of such cells and compositions for reducing tumor burden in a subject, lengthening survival of a subject having a neoplasia, a pathogen infection, and/or an autoimmune disorder, treating and/or preventing neoplasia or other diseases/disorders, treating and/or preventing autoimmune diseases, and/or reducing and/or preventing an antibody-mediated rejection of cells and/or tissues in a subject, e.g., a subject receives an organ transplant or a cell therapy wherein the cells and/or tissues are used in the cell therapy.

The presently disclosed subject matter is based, at least in part, on the discovery that an IgG-degrading enzyme, e.g., IdeS, can deliver and cleave an IgG, thereby increasing the resistance of the cells to host humoral responses, which lead to prolonged persistence of the cells and more potent activities (e.g., anti-tumor activities) of the cells. The prolonged persistence of the cells can also improve the cost effectiveness of therapies including such cells, e.g., CAR-T cell therapies.

Non-limiting embodiments of the presently disclosed subject matter are described by the present specification and Examples.

For purposes of clarity of the presently disclosed subject matter and not by way of limitation, the detailed description is divided into the following subsections:
5.1. Definitions;
5.2. IgG-degrading Enzymes;
5.3. Antigen Ligand-Recognizing Receptors
5.4. Cells;
5.5. Compositions and Vectors;
5.6. Polypeptides and Analogs;
5.7. Administration;
5.8. Formulations;
5.9. Methods of Uses; and
5.10. Kits.

### 5.1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

By “immunoresponsive cell” is meant a cell that functions in an immune response or a progenitor, or progeny thereof. In certain embodiments, the immunoresponsive cell is a cell of the lymphoid lineage or a cell of the myeloid lineage. Non-limiting examples of cells of lymphoid lineage include T cells, Natural Killer (NK) cells, dendritic cells, B cells, and stem cells (e.g., induced pluripotent stem cells) from which lymphoid cells may be differentiated. Non-limiting examples of cells of the myeloid lineage include monocytes, macrophages, neutrophils, basophils, eosinophils, erythrocytes, megakaryocytes, and stem cells from which myeloid cells may be differentiated.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ϵ/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immune or immunoresponsive cell (e.g., a T-cell) in response to its binding to an antigen.

Antigen-binding fragments include F(ab′)₂, and Fab. F(ab′)₂, and Fab fragments that lack the Fc fragment of an intact antibody.

In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242).

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27 (6):455-51; Peter et al., J Cachexia Sarcopenia Muscle 2012 Aug. 12; Shieh et al., J Imuno 12009 183 (4):2277-85; Giomarelli et al., Thromb Haemost 2007 97 (6):955-63; Fife eta., J Clin Invst 2006 116 (8):2252-61; Brocks et al., Immunotechnology 1997 3 (3):173-84; Moosmayer et al., Ther Immunol 1995 2 (10:31-40). Agonistic scFvs having stimulatory activity have been described (see, e.g., Peter et al., J Bioi Chern 2003 25278 (38):36740-7; Xie et al., Nat Biotech 1997 15 (8):768-71; Ledbetter et al., Crit Rev Immuno11997 17 (5-6):427-55; Ho et al., BioChim Biophys Acta 2003 1638 (3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immune or immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain.

As used herein, the term “nucleic acid molecules” include any nucleic acid molecule that encodes a polypeptide of interest (e.g., an IL-36 polypeptide) or a fragment thereof. Such nucleic acid molecules need not be 100% homologous or identical with an endogenous nucleic acid sequence, but may exhibit substantial identity. Polynucleotides having “substantial identity” or “substantial homology” to an endogenous sequence are typically capable of hybridizing with at least one strand of a double-stranded nucleic acid molecule. By “hybridize” is meant a pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, e.g., less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, e.g., at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% SDS. In certain embodiments, hybridization will occur at 37° C. in 500 mM NaCl, 50 mM trisodium citrate, 1% SDS, 35% formamide, and 100 μg/m1 denatured salmon sperm DNA (ssDNA). In certain embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, e.g., less than about 15 mM NaCl and 1.5 mM trisodium citrate. Stringent temperature conditions for the wash steps will ordinarily include a temperature of at least about 25° C., of at least about 42° C., or of at least about 68° C. In certain embodiments, wash steps will occur at 25° C. in 30 mM NaCl, 3 mM trisodium citrate, and 0.1% SDS. In certain embodiments, wash steps will occur at 42° C. in 15 mM NaCl, 1.5 mM trisodium citrate, and 0.1% SDS. In certain embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” or “substantially homologous” is meant a polypeptide or a polynucleotide exhibiting at least about 50% homologous or identical to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the amino acid sequence or the nucleic acid sequence used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “analog” is meant a structurally related polypeptide or nucleic acid molecule having the function of a reference polypeptide or nucleic acid molecule.

The term “ligand” as used herein refers to a molecule that binds to a receptor. In certain embodiments, the ligand binds to a receptor on another cell, allowing for cell-to-cell recognition and/or interaction.

The term “constitutive expression” or “constitutively expressed” as used herein refers to expression or expressed under all physiological conditions.

By “disease” is meant any condition, disease or disorder that damages or interferes with the normal function of a cell, tissue, or organ, e.g., neoplasia, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “enforcing tolerance” is meant preventing the activity of self-reactive cells or immunoresponsive cells that target transplanted organs or tissues.

By “endogenous” is meant a polynucleotide or a polypeptide that is normally expressed in a cell or a tissue.

By “exogenous” is meant a polynucleotide or a polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasia” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells).

By “receptor” is meant a polypeptide or a portion or fragment thereof, present on a cell membrane that selectively binds to at least one ligand. In certain embodiments, the ligand is an antigen. The antigen can be a tumor antigen, a pathogen antigen, or a normal cell antigen. an HLA antigen, or an alloantigen (e.g., a minor histocompatibility alloantigen).

By “recognize” is meant selectively binds to a target, e.g., a ligand (e.g., an antigen). For example, a cell (e.g., a T cell) that recognizes a tumor can expresses a receptor (e.g., a TCR or a CAR) that binds to a tumor antigen.

As used herein, the term “ligand-recognzing receptor” refers to a receptor that is capable of recognizing a ligand.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell, e.g., via the secretory pathway through the endoplasmic reticulum, Golgi apparatus, and as a vesicle that transiently fuses at the cell plasma membrane, releasing the polypeptide outside of the cell.

By “specifically binds” is meant a polypeptide or a fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-IS neoplastic cell. In certain embodiments, a tumor antigen includes any polypeptide expressed by a tumor that is capable of activating or inducing an immune response via an antigen-recognizing receptor (e.g., CD19, MUC-16) or capable of suppressing an immune response via receptor-ligand binding (e.g., CD47, PD-L1/L2, B7.1/2).

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

Other aspects of the presently disclosed subject matter are described in the following disclosure and are within the ambit of the presently disclosed subject matter.

### 5.2. IgG-Degrading Enzymes

The presently disclosed cells comprise an IgG-degrading enzyme.

The IgG-degrading enzyme is capable of cleaving an IgG. IgG plays an important protective role in the human immune system, but is also associated in the pathogenesis of diseases such as rheumatoid arthritis, myasthenia gravis, systemic lupus etc., where removal of IgG has been used as a therapeutic avenue to treat these autoimmune diseases (Johansson et al., PLoS ONE (2008); 3:1-6; Berta et al., The International Journal of Artificial Organs (1994); 17:603-608, Stummvoll et al., Annals of the Rheumatic Diseases (2005); 64:1015-1021). In addition, host IgG plays an important role in allotransplants, where incompatibility between HLA donors leads to antibody-mediated rejection of allografts (Loupy et al., New England Journal of Medicine (2018); 379:1150-1160).

IdeS was evaluated in humans for desensitization prior to allotransplants. In the study, 24 out of 25 patients were able to receive HLA-incompatible transplants, after treatment with IdeS which rapidly removed all donor-specific antibodies (Jordan et al., New England Journal of Medicine (2017); 377:442-453; Lonze et al., Annals of Surgery (2018); 268:488-496).

Studies have shown that IgG-degrading enzymes have positive therapeutic outcomes. For example, IdeS has been shown to have positive therapeutic outcomes in animal models of idiopathic thrombocytopenia, Goodpasture's disease, and arthritis (Johansson et al., PLoS ONE (2008); 3:1-6; Yang et al., Nephrology Dialysis Transplantation (2010); 25:2479-2486; Nandakumar et al., Arthritis and Rheumatism (2007); 56:3253-3260. s

The IgG-degrading enzyme can cleave an IgG, thereby preventing an IgG antibody from killing the cell. Additionally or alternatively, the IgG-degrading enzyme can cleave an IgG, thereby allowing the remaining fragment of the IgG to retain the binding to the cell, which protects the cell from one or more cytotoxic antibodies. In certain embodiments, the one or more cytotoxic antibodies bind to the same epitope region as the IgG or cross-compete for binding to the same epitope region with the IgG, thereby killing the cell. Therefore, the process creates a protective shield.

IgG-degrading enzymes can be used to protect cells comprising a ligand-recognizing receptor (e.g., a CAR or a TCR) from host humoral responses. Non-limiting examples of host humoral responses include antibody-driven host immune response (e.g., anti-CAR antibodies), host humoral responses directed to new amino acid sequences, host humoral responses foreign sequences, host humoral responses to fusion point sequences, host humoral responses to alloantigens (e.g., minor histocompatibility alloantigens), host humoral responses to HLA antigens, host humoral responses to other allelic, host humoral responses to protein or carbohydrate expression changes, host humoral responses to post-translational modifications of proteins, host humoral responses to derived by the differences between the host and the infused cells. This may include preexisting responses or responses stimulated by the infusion of the cells. The protection from host humoral responses prevents death or neutralization of the cells, provides the cells with increased persistence, improved activities (e.g., anti-tumor activities, proliferation, secretion of cytokines, cytolytic engagement, or other functions specifically engineered into the cell. The increased persistence and function of the cells can also lead to reduced cost for any therapies comprising the cells. For example, CAR-T cell therapy is associated with very high cost, e.g., one-time infusion is upwards of several hundred thousand dollars (Lin, et al., Journal of Clinical Oncology (2018); 36:3192-3202). Improving the persistence of CAR-T cells can improve the cost effectiveness of this type of treatment.

Non-limiting examples of IgG-degrading enzymes include IgG-degrading enzyme of S. pyogenes (e.g. IdeS), IgG-degrading enzyme of S. equi subsp. zooepidemicus (IdeZ), IgG-degrading enzyme of S. equi subsp. equi. (IdeE), an endoglycosidase from Streptococcus pyogenes (EndoS), and a streptococcal cysteine proteinase from Streptococcus pyogenes (SpeB).

IdeE and IdeZ are derived from Streptococcus equi (Lannergard et al., FEMS Microbiology Letters (2006); 262:230-235). Each of IdeE and IdeZ cleaves the Fc region below the hinge region of an IgG, wherein the region comprises a site LLGGP.

EndoS is an endoglycosidase that removes the glycan moiety on the gamma-chains of an IgG, thereby interfering the interaction of IgG with Fc receptors (Collin et al., EMBO J. (2001); 20(12):3046-3055.

In certain embodiments, the IgG-degrading enzyme is capable of interfering the interaction between IgG and Fc receptors. In certain embodiments, the IgG-degrading enzyme is an endopeptidase, e.g., IdeS, IdeZ, IdeE, and SpeB. In certain embodiments, the IgG-degrading enzyme is an IgG specific endopeptidase, e.g., IdeS, IdeZ, and IdeE. In certain embodiments, the IgG-degrading enzyme is an endoglycosidase, e.g., EndoS.

In certain embodiments, the IgG-degrading enzyme is IdeS. Bacteria have evolved intricate strategies to evade the human immune system, such as the release of proteolytic enzymes to avoid opsonization and phagocytosis (Potempa et al., Biol Chem. (2012); 393:873-888). Streptococcus pyogenes secretes an IgG-degrading enzyme, which cleaves IgG below the hinge region resulting in Fab and Fc fragments.

IdeS is a cysteine protease with high specificity for immunoglobulin G, that does not cleave immunoglobulins A, M, E and D (Von et al., EMBO Journal (2002); 21:1607-1615, and Johansson et al., PLoS ONE (2008); 3:1-6). While IdeS is itself potentially immunogenic, the enzyme should also protect itself from the host immune response, which is its natural goal. IdeS cleaves an IgG below the hinge region, thereby releasing Fc fragments and the F(ab′)₂ fragments remain intact (von Pawel-Rammingen et al., EMBO J. (2002); 21(7):1607-15).

In certain embodiments, the IdeS has an amino acid sequence that is at least about 80%, at least about 85%, at least about 90%, at least about 95%, at least about 96%, at least about 97%, at least about 98%, at least about 99% or at least about 100% homologous or identical to the amino acid sequence having a GenBank No: AEJ35177.1 (SEQ ID NO: 1) (homology and identity herein may be determined using standard software such as BLAST or FASTA) as provided below, or fragments thereof, and/or may optionally comprise up to one or up to two or up to three conservative amino acid substitutions. In certain embodiments, the IdeS comprises or has an amino acid sequence that is a consecutive portion of SEQ ID NO: 1, which is at least about 20, or at least about 30, or at least about 40, or at least about 50, or at least about 60, or at least about 70, or at least about 100, or at least about 200, or at least about 300, and up to 341 amino acids in length. Alternatively or additionally, in non-limiting various embodiments, the IdeS comprises or has an amino acid sequence of amino acids 1 to 341, 30 to 341, 1 to 50, 50 to 100, 100 to 150, 150 to 200, or 200 to 341 of SEQ ID NO: 1. In certain embodiments, the IdeS comprises or has amino acids 30 to 341 of SEQ ID NO: 1. SEQ ID NO: 1 is provided below.

| [SEQ ID NO: 1] | |
|---|---|
| 1 | MRKRCYSTSA VVLAAVTLFA LSVDRGVIAD SFSANQEIRY SEVTPYHVTS VWTKGVTPPA |
| 61 | KFTQGEDVFH APYVANQGWY DITKTFNGKD DLLCGAATAG NMLHWWFDQN KEKIEAYLKK |
| 121 | HPDKQKIMFG DQELLDVRKV INTKGDQTNS ELFNYFRDKA FPGLSARRIG VMPDLVLDMF |
| 181 | INGYYLNVYK TQTTDVNRTY QEKDRRGGIF DAVFTRGDQS KLLTSRHDFK EKNLKEISDL |
| 241 | IKKELTEGKA LGLSHTYANV RINHVINLWG ADFDSNGNLK AIYVTDSDSN ASIGMKKYFV |
| 301 | GVNSAGKVAI SAKEIKEDNI GAQVLGLFTL STGQDSWNQT N |

An exemplary nucleic acid sequence encoding amino acids 30 to 341 of SEQ ID NO: 1 is set forth in SEQ ID NO: 2, which is provided below.

[SEQ ID NO: 1]
GACTCTTTTAGTGCCAATCAAGAAATCCGATATAGCGAGGTGACTCCTTA
CCATGTAACTTCTGTGTGGACCAAGGGAGTTACCCCACCAGCCAAGTTCA
CGCAGGGTGAGGACGTATTTCACGCACCGTACGTAGCTAACCAGGGTTGG
TACGACATCACTAAGACCTTCAATGGGAAAGACGATCTTTTGTGTGGTGC
CGCAACGGCGGGCAACATGCTGCACTGGTGGTTCGACCAAAACAAGGAGA
AGATCGAAGCGTACTTGAAGAAACACCCAGACAAACAGAAAATCATGTTT
GGAGACCAGGAGCTCCTGGATGTGAGAAAGGTAATCAACACTAAAGGTGA
CCAAACAAACAGTGAACTTTTTAACTATTTTCGGGACAAGGCGTTTCCAG
GATTGAGTGCCAGAAGAATCGGCGTAATGCCTGACCTCGTGCTTGACATG
TTCATCAATGGATACTATCTCAATGTATATAAGACCCAAACCACAGATGT
TAATCGAACTTATCAGGAGAAGGATAGAAGGGGAGGAATATTTGATGCCG
TTTTTACTCGAGGAGACCAGTCTAAGCTCTTGACCAGCAGGCACGACTTC
AAAGAGAAGAATCTTAAAGAAATATCTGATCTCATAAAGAAGGAACTGAC
GGAAGGCAAAGCGCTGGGACTTTCCCATACGTATGCCAATGTAAGAATCA
ATCATGTCATAAACCTTTGGGGTGCTGATTTCGATTCTAATGGAAATCTT
AAGGCTATATATGTTACTGATTCCGACTCCAACGCGTCTATTGGCATGAA
AAAATACTTCGTCGGGGTGAACTCAGCAGGAAAGGTCGCAATATCTGCTA
AGGAAATTAAGGAAGACAACATAGGGGCGCAAGTGCTGGGTCTCTTCACC
CTTTCCACCGGCCAAGACTCCTGGAATCAAACAAAT

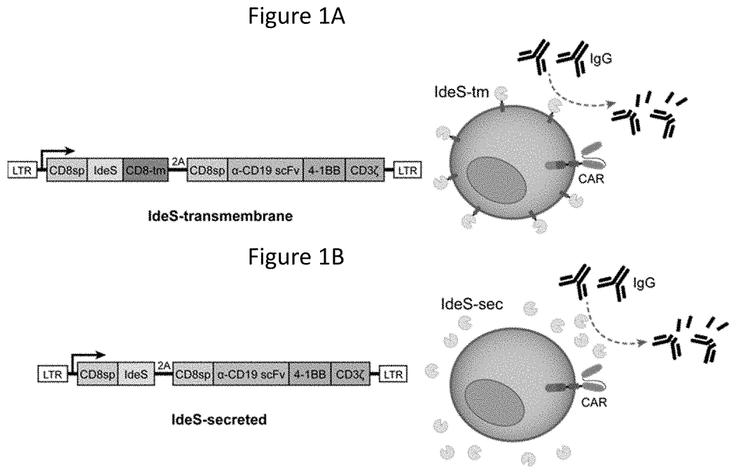

In certain embodiments, the IgG-degrading enzyme is bound to the cells (also referred to as “membrane-bound IgG-degrading enzyme”). See e.g., FIG. 1A. For the membrane-bound IgG-degrading enzyme, the enzyme is fused or attached to a transmembrane domain, which is capable of binding or attaching the enzyme to the cells. See e.g., FIG. 1A. The transmembrane domain can be attached to the C-terminus or the N-terminus of the IgG-degrading enzyme. In certain embodiments, the transmembrane domain is attached to the C-terminus of the IgG-degrading enzyme. See e.g., FIG. 1A.

The transmembrane domain can be a transmembrane domain of a molecule or protein or a portion thereof. The transmembrane domain can comprise a CD8 polypeptide (e.g., the transmembrane domain of CD8 or a portion thereof), a CD28 polypeptide (e.g., the transmembrane domain of CD28 or a portion thereof), a CD3ζ polypeptide (e.g., the transmembrane domain of CD3ζ or a portion thereof), a CD4 polypeptide (e.g., the transmembrane domain of CD4 or a portion thereof), a 4-1BB polypeptide (e.g., the transmembrane domain of 4-1BB or a portion thereof), an OX40 polypeptide (e.g., the transmembrane domain of OX40 or a portion thereof, an ICOS polypeptide (e.g., the transmembrane domain of ICOS or a portion thereof, a synthetic peptide (not based on a protein associated with the immune response), or a combination thereof.

In certain embodiments, the transmembrane domain fused to the IgG-degrading enzyme is a CD8 polypeptide. In certain embodiments, the CD8 polypeptide comprises or has the amino acid sequence set forth in SEQ ID NO: 3 or amino acids 137 to 207 of SEQ ID NO: 27. SEQ ID NO:3 is provided below.

[SEQ ID NO: 3]
PTTTPAPRPPTPAPTIASQPLSLRPEACRPAAGGAVHTRGLDFACDIYIW
APLAGTCGVLLLSLVITLYCN

An exemplary nucleic acid sequence encoding the amino acid of SEQ ID NO: 3 is set forth in

SEQ ID NO: 4, which is provided below.

[SEQ ID NO: 4]
CCAACTACTACTCCCGCACCGAGACCGCCCACTCCTGCTCCCACGATTGC
CTCCCAACCTCTTAGCTTGAGACCGGAAGCATGTCGGCCTGCGGCCGGTG
GCGCAGTACATACTCGCGGCCTGGACTTTGCGTGCGACATATACATTTGG
GCACCCCTGGCCGGCACTTGCGGAGTTTTGCTGCTGTCTCTCGTGATAAC
TCTCTATTGTAAC

In certain embodiments, the IgG-degrading enzyme is secreted from the cells (also referred to as “secreted IgG-degrading enzyme”). See e.g., FIG. 1B. For the secreted IgG-degrading enzyme, the enzyme is not fused or attached to a transmembrane domain, thereby the enzyme is secreted or released from the cells to the extracellular environment or the vicinity of the cells. See e.g., FIG. 1B.

In certain embodiments, the IgG-degrading enzyme is connected or fused to a signal peptide (also referred to as “leader sequence). As used herein, a “signal sequence” or a “leader sequence” refers to a peptide sequence (e.g., about 5, 10, 15, 20, 25 or 30 amino acids) present at the N-terminus of a polypeptide or a protein or a fragment thereof to direct its transportation, e.g., to transport the IgG-degrading enzyme to the cell membrane, or to transport the ligand-recognizing receptor (e.g., a CAR) to the cell membrane.

Exemplary signal sequences include, but are not limited to, a CD4 signal peptide, an IgG heavy chain signal peptide, an IL-2 signal sequence (e.g., a human IL-2 signal peptide having the amino acid sequence set forth in SEQ ID NO: 5 or a mouse IL-2 signal peptide having the amino acid sequence set forth in SEQ ID NO: 6), a kappa signal sequence (e.g., a human kappa signal sequence having the amino acid sequence set forth in SEQ ID NO: 7 or a mouse kappa signal sequence having the amino acid sequence set forth in SEQ ID NO: 8, a CD8 signal sequence (e.g., a human CD8 signal peptide having the amino acid sequence set forth in SEQ ID NO: 9 or a truncated human CD8 signal peptide having the amino acid sequence set forth in SEQ ID NO: 10), an albumin signal sequence (e.g.,: a human albumin signal sequence having the amino acid sequence set forth in SEQ ID NO: 11), and a prolactin signal sequence (e.g., a human prolactin signal sequence having the amino acid sequence set forth in SEQ ID NO: 12). SEQ ID NOS: 5-12 are provided below.

[SEQ ID NO: 5]
MYRMQLLSCIALSLALVTNS
[SEQ ID NO: 6]
MYSMQLASCVTLTLVLLVNS
[SEQ ID NO: 7]
METPAQLLFLLLLWLPDTTG
[SEQ ID NO: 8]
METDTLLLWVLLLWVPGSTG
[SEQ ID NO: 9]
MALPVTALLLPLALLLHAARP
[SEQ ID NO: 10]
MALPVTALLLPLALLLHA
[SEQ ID NO: 11]
MKWVTFISLLFSSAYS
[SEQ ID NO: 12]
MDSKGSSQKGSRLLLLLVVSNLLLCQGVVS

In certain embodiments, the IgG-degrading enzyme is connected or fused to a CD8 signal sequence. In certain embodiments, the CD8 signal sequence comprises or has the amino acid sequence set forth in SEQ ID NO: 10.

An exemplary nucleic acid sequence encoding the amino acid sequence of SEQ ID NO: 10 is set forth in SEQ ID NO: 13, which is provided below.

[SEQ ID NO: 13]
ATGGCCCTTCCGGTGACGGCGCTTCTCCTCCCTTTGGCGCTTCTTCTGCA
CGCT

In certain embodiments, the IgG-degrading enzyme is expressed from a vector. Expression of the IgG-degrading enzyme can be detected by any suitable methods, including, but not limited to, immunoblot, PCR, ELISA, mass spectrometry, and flow cytometry.

### 5.3. Ligand-Recognizing Receptors

The presently disclosed cells comprise a provides ligand-recognizing receptor. Any receptor that is capable of binding to a ligand can be a ligand-recognizing receptor of the present disclosure. Non-limiting examples of ligand-recognizing receptors include antigen-recognizing receptor that bind to an antigen of interest, cell adhesion molecules, cytokine receptors (e.g., interleukin or cytokine receptors, such as Fas ligand or TGFβ receptors, Trail, TCR, IgG, CAR, NK inhibitory receptors, Growth factor receptors such as EGFR or FGFR, peptide ligands or adhesion molecules, carbohydrate receptors, G protein receptors, etc.), and Fc receptors. Receptors can be monovalent or multivalent. The ligand-recognizing receptor can be endogenous or exogenous. The ligand-recognizing receptor can be recombinantly expressed. In certain embodiments, the ligand-recognizing receptor is expressed from a vector.

In certain embodiments, the ligand-recognizing receptor is an antigen-recognizing receptor that bind to an antigen of interest. Non-limiting examples of antigen-recognizing receptors include chimeric antigen receptors (CARs), T-cell receptors (TCRs), IgG, B cell receptors (BCR), IgM, IgD, and IgE.

In certain embodiments, the ligand-recognizing receptor is a chimeric antigen receptors (CARs). In certain embodiments, the ligand-recognizing receptor is a T-cell receptor (TCR).

In certain embodiments, the ligand-recognizing receptor binds to an antigen. The antigen can be a tumor antigen, a pathogen antigen, a normal cell antigen (e.g., for autoimmune diseases or organ transplant), an HLA antigen, or an alloantigen (e.g., a minor histocompatibility alloantigen).

### 5.3.1. Antigens

In certain embodiments, the ligand-recognizing receptor binds to an antigen, which is a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include carbonic anhydrase IX (CA1X), carcinoembryonic antigen (CEA), CD2, CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), HPV E6 or E7 peptides, EBV peptides, MAGE peptide, epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1,GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), and Wilms tumor protein (WT-1), BCMA, NKCS1, EGF1R, EGFR-VIII, CD99, CD70, ADGRE2, CCR1, LILRB2, PRAME, and ERBB.

In certain embodiments, the ligand-recognizing receptor binds to CD19. In certain embodiments, the ligand-recognizing receptor binds to a murine CD19 polypeptide. In certain embodiments, the murine CD19 polypeptide comprises the amino acid sequence set forth in SEQ ID NO: 14.

[SEQ ID NO: 14]
RPQKSLLVEVEEGGNVVLPCLPDSSPVSSEKLAWYRGNQSTPFLELSPGS
PGLGLHVGSLGILLVIVNVSDHMGGFYLCQKRPPFKDIWQPAWTVNVEDS
GEMFRWNASDVRDLDCDLRNRSSGSHRSTSGSQLYVWAKDHPKVWGTKPV
CAPRGSSLNQSLINQDLTVAPGSTLWLSCGVPPVPVAKGSISWTHVHPRR
PNVSLLSLSLGGEHPVREMWVWGSLLLLPQATALDEGTYYCLRGNLTIER
HVKVIARSAVWLWLLRTGG

In certain embodiments, the ligand -recognizing receptor binds to a human CD19 polypeptide. In certain embodiments, the human CD19 polypeptide comprises the amino acid sequence set forth in SEQ ID NO: 15.

[SEQ ID NO: 15]
PEEPLVVKVEEGDNAVLQCLKGTSDGPTQQLTWSRESPLKPFLKLSLGLP
GLGIHMRPLAIWLFIFNVSQQMGGFYLCQPGPPSEKAWQPGWTVNVEGSG
ELFRWNVSDLGGLGCGLKNRSSEGPSSPSGKLMSPKLYVWAKDRPEIWEG
EPPCLPPRDSLNQSLSQDLTMAPGSTLWLSCGVPPDSVSRGPLSWTHVHP
KGPKSLLSLELKDDRPARDMWVMETGLLLPRATAQDAGKYYCHRGNLTMS
FHLEITARPVLWHWLLRTGGWK

In certain embodiments, the ligand -recognizing receptor binds to the extracellular domain of a human or murine CD19 protein.

In certain embodiments, the ligand -recognizing receptor binds to a pathogen antigen, e.g., for use in treating and/or preventing a pathogen infection or other infectious disease, for example, in an immunocompromised subject. Non-limiting examples of pathogen includes a virus, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtherias, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), a viral antigen present in human papillomavirus (HPV), or a viral antigen present in influenza virus.

In certain embodiments, the ligand -recognizing receptor binds to an alloantigen, such as an HLA molecule, and a minor histocompatibility alloantigen.

### 5.3.2. T-Cell Receptor (TCR)

In certain embodiments, the ligand-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each has three complementarity determining regions (CDRs).

In certain embodiments, a TCR can form a receptor complex with three dimeric signaling modules CD3δ/ϵ, CD3γ/ϵ and CD247 ζ/ζ or ζ/η. When a TCR complex engages with its antigen and MHC (peptide/MHC), the T cell expressing the TCR complex is activated.

In certain embodiments, the ligand-recognizing receptor is an endogenous TCR. In certain embodiments, the ligand-recognizing receptor is an exogenous TCR. In certain embodiments, the ligand-recognizing receptor is a recombinant TCR. In certain embodiments, the ligand-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 5.3.3. Chimeric Antigen Receptor (CAR)

In certain embodiments, the ligand-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., a scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first generation CAR. In certain embodiments, the antigen-recognizing receptor is a second generation CAR.

In certain non-limiting embodiments, the extracellular antigen-binding domain of the CAR (embodied, for example, an scFv or an analog thereof) binds to an antigen with a dissociation constant (K_{d}) of about 5×10⁻⁷ M or less. In certain embodiments, the K_{d} is about 5×10⁻⁷ or less, about 1×10⁻⁷ M or less, about 5×10⁻⁸ M or less, about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, about 1×10⁻¹¹ M or less, about 5×10⁻¹² M or less, or about 1×10⁻¹² M or less.

Binding of the extracellular antigen-binding domain (for example, in an scFv or an analog thereof) can be confirmed by, for example, enzyme-linked immunosorbent assay (ELISA), radioimmunoassay (RIA), FACS analysis, bioassay (e.g., growth inhibition), surface plasmon resonance, Western Blot assay, other assays known in the art. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

In accordance with the presently disclosed subject matter, a CARs can comprise an extracellular antigen-binding domain, a transmembrane domain and an intracellular signaling domain, wherein the extracellular antigen-binding domain specifically binds to an antigen, e.g., a tumor antigen or a pathogen antigen.

### 5.3.3.1. Extracellular Antigen Binding Domain of A CAR

In certain embodiments, the extracellular antigen-binding domain of the CAR comprises a scFv. In certain embodiments, the scFv is a human scFv. In certain embodiments, the scFv is a humanized scFv. In certain embodiments, the scFv is a murine scFv. In certain embodiments, the extracellular antigen-binding domain of the CAR comprises a Fab, which is optionally crosslinked. In certain embodiments, the extracellular antigen-binding domain of the CAR comprises a F(ab)₂. In certain embodiments, any of the foregoing molecules may be comprised in a fusion protein with a heterologous sequence to form the extracellular antigen-binding domain of the CAR. In certain embodiments, the extracellular antigen-binding domain of the CAR comprises a murine scFv. In certain embodiments, the extracellular antigen-binding domain of a presently disclosed CAR comprises a scFv that binds to CD19.

In certain embodiments, the scFv comprises a heavy chain variable region (V_{H}) comprising the amino acid sequence set forth in SEQ ID NO: 16. In certain embodiments, the scFv comprises a light chain variable region (V_{L}) comprising the amino acid sequence set forth in SEQ ID NO: 17. In certain embodiments, the scFv comprises a V_{H} comprising the amino acid sequence set forth in SEQ ID NO: 16 and a V_{L} comprising the amino acid sequence set forth in SEQ ID NO: 17, optionally with (iii) a linker sequence, for example a linker peptide, between the V_{H} and the V_{L}.

“Linker”, as used herein, refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains).

In certain embodiments, the linker comprises the amino acid sequence set forth in SEQ ID NO: 18, which is provided below.

[SEQ ID NO: 18]
GGGGSGGGGSGGGGS

In certain embodiments, the extracellular antigen-binding domain of the CAR comprises a V_{H} comprising an amino acid sequence that is at least about 80% (e.g., at least about 85%, at least about 90%, or at least about 95%) homologous or identical to SEQ ID NO: 16. For example, the extracellular antigen-binding domain of the CAR comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 16. In certain embodiments, the extracellular antigen-binding domain of the CAR comprises a V_{H} comprising the amino acid sequence set forth in SEQ ID NO: 16.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to SEQ ID NO: 17. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 17. In certain embodiments, the extracellular antigen-binding domain of the CAR comprises a V_{L} comprising the amino acid sequence set forth in SEQ ID NO: 17. In certain embodiments, the extracellular antigen-binding domain of the CAR comprises a V_{H} comprising an amino acid sequence that is at least about 80% (e.g., at least about 85%, at least about 90%, or at least about 95%) homologous or identical to SEQ ID NO: 16, and a V_{L} comprising an amino acid sequence that is at least about 80% (e.g., at least about 85%, at least about 90%, or at least about 95%) homologous or identical to SEQ ID NO: 17. In certain embodiments, the extracellular antigen-binding domain of the CAR comprises a V_{H} comprising the amino acid sequence set forth in SEQ ID NO: 16 and a V_{L} comprising the amino acid sequence set forth in SEQ ID NO: 17.

In certain embodiments, the extracellular antigen-binding domain of the CAR comprises a V_{H} CDR1 comprising the amino acid sequence set forth in SEQ ID NO: 19 or a conservative modification thereof, a V_{H} CDR2 comprising the amino acid sequence set forth in SEQ ID NO: 20 or a conservative modification thereof, and a V_{H} CDR3 comprising the amino acid sequence set forth in SEQ ID NO: 21 a conservative modification thereof In certain embodiments, the extracellular antigen-binding domain of the CAR comprises a V_{H} CDR1 comprising the amino acid sequence set forth in SEQ ID NO: 19, a V_{H} CDR2 comprising the amino acid sequence set forth in SEQ ID NO: 20, and a V_{H} CDR3 comprising the amino acid sequence set forth in SEQ ID NO: 21. In certain embodiments, the extracellular antigen-binding domain of the CAR comprises a V_{L} CDR1 comprising the amino acid sequence set forth in SEQ ID NO: 22 or a conservative modification thereof, a V_{L} CDR2 comprising the amino acid sequence set forth in SEQ ID NO: 23 or a conservative modification thereof, and a V_{L} CDR3 comprising the amino acid sequence set forth in SEQ ID NO: 24 or a conservative modification thereof. In certain embodiments, the extracellular antigen-binding domain of the CAR comprises a V_{L} CDR1 comprising the amino acid sequence set forth in SEQ ID NO: 22, a V_{L} CDR2 comprising the amino acid sequence set forth in SEQ ID NO: 23, and a V_{L} CDR3 comprising the amino acid sequence set forth in SEQ ID NO: 24. In certain embodiments, the extracellular antigen-binding domain of the CAR comprises a V_{H} CDR1 comprising the amino acid sequence set forth in SEQ ID NO: 19 or a conservative modification thereof, a V_{H} CDR2 comprising the amino acid sequence set forth in SEQ ID NO: 20 or a conservative modification thereof, a V_{H} CDR3 comprising the amino acid sequence set forth in SEQ ID NO: 21 a conservative modification thereof, a V_{L} CDR1 comprising the amino acid sequence set forth in SEQ ID NO: 22 or a conservative modification thereof, a V_{L} CDR2 comprising the amino acid sequence set forth in SEQ ID NO: 23 or a conservative modification thereof, and a V_{L} CDR3 comprising the amino acid sequence set forth in SEQ ID NO: 24 or a conservative modification thereof. In certain embodiments, the extracellular antigen-binding domain of the CAR comprises a V_{H} CDR1 comprising amino acids having the sequence set forth in SEQ ID NO: 19, a V_{H} CDR2 comprising the amino acid sequence set forth in SEQ ID NO: 20, a V_{H} CDR3 comprising the amino acid sequence set forth in SEQ ID NO: 21, a V_{L} CDR1 comprising the amino acid sequence set forth in SEQ ID NO: 22, a V_{L} CDR2 comprising the amino acid sequence set forth in SEQ ID NO: 23, and a V_{L} CDR3 comprising the amino acid sequence set forth in SEQ ID NO: 24.

In certain embodiments, the extracellular antigen-binding domain comprises a scFv comprising the amino acid sequence of SEQ ID NO: 25 and specifically binds to a human CD19 polypeptide (e.g., a human CD19 polypeptide comprising the amino acid sequence set forth in SEQ ID NO: 15). In certain embodiments, the nucleotide sequence encoding the amino acid sequence of SEQ ID NO: 25 is set forth in SEQ ID NO: 26.

SEQ ID Nos: 16, 17, and 19-26 are provided below.

[SEQ ID NO: 16]
EVKLQQSGAELVRPGSSVKISCKASGYAFSSYWMNWVKQRPGQGLEWIGQ
IYPGDGDTNYNGKFKGQATLTADKSSSTAYMQLSGLTSEDSAVYFCARKT
ISSVVDFYFDYWGQGTTVTVSS
[SEQ ID NO: 17]
DIELTQSPKFMSTSVGDRVSVTCKASQNVGTNVAWYQQKPGQSPKPLIYS
ATYRNSGVPDRFTGSGSGTDFTLTITNVQSKDLADYFCQQYNRYPYTSGG
GTKLEIKR
[SEQ ID NO: 19]
GYAFSSY
[SEQ ID NO: 20]
YPGDGD
[SEQ ID NO: 21]
KTISSVVDFYFDY
[SEQ ID NO: 22]
KASQNVGTNVA
[SEQ ID NO: 23]
SATYRNS
[SEQ ID NO: 24]
QQYNRYPYT
[SEQ ID NO: 25]
EVKLQQSGAELVRPGSSVKISCKASGYAFSSYWMNWVKQRPGQGLEWIGQ
IYPGDGDTNYNGKFKGQATLTADKSSSTAYMQLSGLTSEDSAVYFCARKT
ISSVVDFYFDYWGQGTTVTVSSGGGGSGGGGSGGGGSDIELTQSPKFMST
SVGDRVSVTCKASQNVGTNVAWYQQKPGQSPKPLIYSATYRNSGVPDRFT
GSGSGTDFTLTITNVQSKDLADYFCQQYNRYPYTSGGGTKLEIKR
[SEQ ID NO: 26]
GAGGTGAAGCTGCAGCAGTCTGGGGCTGAGCTGGTGAGGCCTGGGTCCTC
AGTGAAGATTTCCTGCAAGGCTTCTGGCTATGCATTCAGTAGCTACTGGA
TGAACTGGGTGAAGCAGAGGCCTGGACAGGGTCTTGAGTGGATTGGACAG
ATTTATCCTGGAGATGGTGATACTAACTACAATGGAAAGTTCAAGGGTCA
AGCCACACTGACTGCAGACAAATCCTCCAGCACAGCCTACATGCAGCTCA
GCGGCCTAACATCTGAGGACTCTGCGGTCTATTTCTGTGCAAGAAAGACC
ATTAGTTCGGTAGTAGATTTCTACTTTGACTACTGGGGCCAAGGGACCAC
GGTCACCGTCTCCTCAGGTGGAGGTGGATCAGGTGGAGGTGGATCTGGTG
GAGGTGGATCTGACATTGAGCTCACCCAGTCTCCAAAATTCATGTCCACA
TCAGTAGGAGACAGGGTCAGCGTCACCTGCAAGGCCAGTCAGAATGTGGG
TACTAATGTAGCCTGGTATCAACAGAAACCAGGACAATCTCCTAAACCAC
TGATTTACTCGGCAACCTACCGGAACAGTGGAGTCCCTGATCGCTTCACA
GGCAGTGGATCTGGGACAGATTTCACTCTCACCATCACTAACGTGCAGTC
TAAAGACTTGGCAGACTATTTCTGTCAACAATATAACAGGTATCCGTACA
CGTCCGGAGGGGGGACCAAGCTGGAGATCAAACGG

As used herein, the term “a conservative sequence modification” refers to an amino acid modification that does not significantly affect or alter the binding characteristics of the presently disclosed CAR (e.g., the extracellular antigen-binding domain of the CAR) comprising the amino acid sequence. Conservative modifications can include amino acid substitutions, additions and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

The V_{H} and/or V_{L} amino acid sequences having at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NOs:16 and 17) may contain substitutions (e.g., conservative substitutions), insertions, or deletions relative to the specified sequence(s), but retain the ability to bind to a target antigen (e.g., CD19). In certain embodiments, a total of 1 to 10 amino acids are substituted, inserted and/or deleted in a specific sequence (e.g., SEQ ID NOs: 16 and 17). In certain embodiments, substitutions, insertions, or deletions occur in regions outside the CDRs (e.g., in the FRs) of the extracellular antigen-binding domain. In certain embodiments, the extracellular antigen-binding domain of the CAR comprises V_{H} and/or V_{L} sequence selected from the group consisting of SEQ ID NOs: 16 and 17, including post-translational modifications of that sequence (SEQ ID NO: 16 and 17).

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the)(BLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

### 5.3.3.2. Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. The transmembrane domain of the CAR can comprise a CD8 polypeptide (e.g., the transmembrane domain of CD8 or a portion thereof), a CD28 polypeptide (e.g., the transmembrane domain of CD28 or a portion thereof), a CD3ζ polypeptide, a CD4 polypeptide (e.g., the transmembrane domain of CD4 or a portion thereof), a 4-1BB polypeptide (e.g., the transmembrane domain of 4-1BB or a portion thereof), an OX40 polypeptide (e.g., the transmembrane domain of OX4 or a portion thereof, an ICOS polypeptide (e.g., the transmembrane domain of ICOS or a portion thereof, a synthetic peptide (not based on a protein associated with the immune response), or a combination thereof.

In certain embodiments, the transmembrane domain of the CAR comprises a CD8 polypeptide, e.g., the transmembrane domain of human CD8 or a portion thereof, or the transmembrane domain of murine CD8. In certain embodiments, the CD8 polypeptide comprises or has an amino acid sequence that is at least about 85%, about 90%, about 95%, about 96%, about 97%, about 98%, about 99% or about 100% homologous or identical to the sequence having a NCBI Reference No: NP_001139345.1 (SEQ ID NO: 27) (homology herein may be determined using standard software such as BLAST or FASTA) as provided below, or fragments thereof, and/or may optionally comprise up to one or up to two or up to three conservative amino acid substitutions. In certain embodiments, the CD8 polypeptide comprises or has an amino acid sequence that is a consecutive portion of SEQ ID NO: 27, which is at least 20, or at least 30, or at least 40, or at least 50, and up to 235 amino acids in length. Alternatively or additionally, in non-limiting various embodiments, the CD8 polypeptide comprises or has an amino acid sequence of amino acids 1 to 235, 1 to 50, 50 to 100, 100 to 150, 137 to 207, 137 to 209, 150 to 200, or 200 to 235 of SEQ ID NO: 25. In certain embodiments, the transmembrane domain of the CAR comprises a CD8 polypeptide comprising or having amino acids 137 to 207 of SEQ ID NO: 27.

[SEQ ID NO: 27]
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

An exemplary nucleotide sequence encoding amino acids 137 to 207 of SEQ ID NO: 27 is set forth in SEQ ID NO: 28, which is provided below.

[SEQ ID NO: 28]
CCCACCACGACGCCAGCGCCGCGACCACCAACCCCGGCGCCCACGATCGC
GTCGCAGCCCCTGTCCCTGCGCCCAGAGGCGTGCCGGCCAGCGGCGGGGG
GCGCAGTGCACACGAGGGGGCTGGACTTCGCCTGTGATATCTACATCTGG
GCGCCCCTGGCCGGGACTTGTGGGGTCCTTCTCCTGTCACTGGTTATCAC
CCTTTACTGCAAC

In certain embodiments, the CD8 polypeptide comprises or has an amino acid sequence that is at least about 85%, about 90%, about 95%, about 96%, about 97%, about 98%, about 99% or about 100% homologous or identical to the sequence having a NCBI Reference No: AAA92533.1 (SEQ ID NO: 29) (homology herein may be determined using standard software such as BLAST or FASTA) as provided below, or fragments thereof, and/or may optionally comprise up to one or up to two or up to three conservative amino acid substitutions. In certain embodiments, the CD8 polypeptide comprises or has an amino acid sequence that is a consecutive portion of SEQ ID NO: 27, which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, 151 to 219, or 200 to 247 of SEQ ID NO: 29. In certain embodiments, the transmembrane domain of the CAR comprises a CD8 polypeptide comprising or having amino acids 151 to 219 of SEQ ID NO: 29. SEQ ID NO: 29 is provided below

| [SEQ ID NO: 29] | |
|---|---|
| 1 | MASPLTRELS LNLLLMGESI ILGSGEAKPQ APELRIFPKK MDAELGQKVD LVCEVLGSVS |
| 61 | QGCSWLFQNS SSKLPQPTFV VYMASSHNKI TWDEKLNSSK LFSAVRDTNN KYVLTLNKFS |
| 121 | KENEGYYFCS VISNSVMYFS SVVPVLQKVN STTTKPVLRT PSPVHPTGTS QPQRPEDCRP |
| 181 | RGSVKGTGLD FACDIYIWAP LAGICVAPLL SLIITLICYH RSRKRVCKCP RPLVRQEGKP |
| 241 | RPSEKIV |

In certain embodiments, the transmembrane domain of the CAR comprises a CD28 polypeptide, e.g., the transmembrane domain of human CD28 or a portion thereof, or the transmembrane domain of murine CD28. The CD28 polypeptide can have an amino acid sequence that is at least about 85%, about 90%, about 95%, about 96%, about 97%, about 98%, about 99% or 100% homologous or identical to the sequence having a NCBI Reference No: NP_006130 (SEQ ID NO: 30), or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 30, which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 150 to 200, or 200 to 220 of SEQ ID NO: 30. In certain embodiments, the transmembrane domain of the CAR comprises a CD28 polypeptide comprising or having amino acids 153 to 179 of SEQ ID NO: 30.

SEQ ID NO: 30 is provided below:

[SEQ ID NO: 30]

| | |
|---|---|
| 1 | MLRLLLALNL FPSIQVTGNK ILVKQSPMLV AYDNAVNLSC |
| | KYSYNLFSRE FRASLHKGLD |
| 61 | SAVEVCVVYG NYSQQLQVYS KTGFNCDGKL GNESVTFYLQ |
| | NLYVNQTDIY FCKIEVMYPP |
| 121 | PYLDNEKSNG TIIHVKGKHL CPSPLFPGPS KPFWVLVVVG |
| | GVLACYSLLV TVAFIIFWVR |
| 181 | SKRSRLLHSD YMNMTPRRPG PTRKHYQPYA PPRDFAAYRS |

In certain non-limiting embodiments, the CAR further comprises a spacer region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and portions of CD3, a portion of a CD28 polypeptide (e.g., a portion of SEQ ID NO: 30), a portion of a CD8 polypeptide (e.g., a portion of SEQ ID NO: 27 or 29), a variation of any of the foregoing which is at least about 80%, at least about 85%, at least about 90%, or at least about 95% homologous or identical thereto, or a synthetic spacer sequence.

### 5.3.3.3. Intracellular Signaling Domain of a CAR

In certain non-limiting embodiments, the intracellular signaling domain of the CAR comprises a CD3ζ polypeptide, which can activate or stimulate a cell (e.g., a cell of the lymphoid lineage, e.g., a T cell). CD3ζ comprises 3 ITAMs, and transmits an activation signal to the cell (e.g., a cell of the lymphoid lineage, e.g., a T cell) after antigen is bound. The intracellular signaling domain of the CD3ζ-chain is the primary transmitter of signals from endogenous TCRs. In certain embodiments, the CD3ζ polypeptide comprises or has an amino acid sequence that is at least about 85%, about 90%, about 95%, about 96%, about 97%, about 98%, about 99% or about 100% homologous to the sequence having a NCBI Reference No: NP_932170 (SEQ ID NO: 31), or fragments thereof, and/or may optionally comprise up to one or up to two or up to three conservative amino acid substitutions. In certain non-limiting embodiments, the CD3ζ polypeptide comprises or has an amino acid sequence that is a consecutive portion of SEQ ID NO: 31, which is at least 20, or at least 30, or at least 40, or at least 50, and up to 164 amino acids in length. Alternatively or additionally, in non-limiting various embodiments, the CD3ζ polypeptide comprises or has an amino acid sequence of amino acids 1 to 164, 1 to 50, 50 to 100, 100 to 150, or 150 to 164 of SEQ ID NO: 31. In certain embodiments, the intracellular signaling domain of the CAR comprises a CD3ζ polypeptide comprising or having amino acids 52 to 164 of SEQ ID NO: 31.

SEQ ID NO: 31 is provided below:

[SEQ ID NO: 31]

| | |
|---|---|
| 1 | MKWKALFTAA ILQAQLPITE AQSFGLLDPK LCYLLDGILF |
| | IYGVILTALF LRVKFSRSAD |
| 61 | APAYQQGQNQ LYNELNLGRR EEYDVLDKRR GRDPEMGGKP |
| | QRRKNPQEGL YNELQKDKMA |
| 121 | EAYSEIGMKG ERRRGKGHDG LYQGLSTATK DTYDALHMQA |
| | LPPR |

In certain embodiments, the CD3ζ polypeptide comprises or has an amino acid sequence that is at least about 85%, about 90%, about 95%, about 96%, about 97%, about 98%, about 99% or about 100% homologous or identical to the sequence having a NCBI Reference No: NP_001106864.2 (SEQ ID NO: 32), or fragments thereof, and/or may optionally comprise up to one or up to two or up to three conservative amino acid substitutions. In certain non-limiting embodiments, the CD3ζ polypeptide comprises or has an amino acid sequence that is a consecutive portion of SEQ ID NO: 32, which is at least about 20, or at least about 30, or at least about 40, or at least about 50, or at least about 90, or at least about 100, and up to 188 amino acids in length. Alternatively or additionally, in non-limiting various embodiments, the CD3ζ polypeptide comprises or has an amino acid sequence of amino acids 1 to 164, 1 to 50, 50 to 100, 52 to 142, 100 to 150, or 150 to 188 of SEQ ID NO: 32. In certain embodiments, the intracellular signaling domain of the CAR comprises a CD3ζ polypeptide comprising or having amino acids 52 to 142 of SEQ ID NO: 32.

SEQ ID NO: 32 is provided below:

[SEQ ID NO: 32]

| | |
|---|---|
| 1 | MKWKVSVLAC ILHVRFPGAE AQSFGLLDPK LCYLLDGILF |
| | IYGVIITALY LRAKFSRSAE |
| 61 | TAANLQDPNQ LYNELNLGRR EEYDVLEKKR ARDPEMGGKQ |
| | RRRNPQEGVY NALQKDKMAE |
| 121 | AYSEIGTKGE RRRGKGHDGL YQDSHFQAVQ FGNRREREGS |
| | ELTRTLGLRA RPKACRHKKP |
| 181 | LSLPAAVS |

In certain embodiments, the intracellular signaling domain of the CAR comprises a CD3ζ polypeptide comprising or having the amino acid sequence set forth in SEQ ID NO: 33, which is provided below.

[SEQ ID NO: 33]
RVKFSRSADAPAYQQGQNQLYNELNLGRREEYDVLDKRRGRDPEMGGKPR
RKNPQEGL

An exemplary nucleic acid sequence encoding SEQ ID NO: 33 is set forth in SEQ ID NO: 34, which is provided below.

[SEQ ID NO: 34]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTG

In certain non-limiting embodiments, the intracellular signaling domain of the CAR further comprises at least a co-stimulatory signaling region. In certain embodiments, the co-stimulatory region comprises at least one co-stimulatory molecule or a portion thereof (e.g., the intracellular domain of a co-stimulatory molecule or a portion thereof). The co-stimulatory signaling region can provide optimal lymphocyte activation to the cells. As used herein, “co-stimulatory molecules” refer to cell surface molecules other than antigen-recognizing receptors or their ligands that are required for an efficient response of immunoresponsive cells to an antigen of interest. Non-limiting examples of co-stimulatory molecules include CD28, 4-1BB, OX40, ICOS, DAP-10, CD27, CD40, CD2, and NKGD2. A co-stimulatory molecule can bind to a co-stimulatory ligand, which is a protein expressed on cell surface that upon binding to its receptor produces a co-stimulatory response, i.e., an intracellular response that effects the stimulation provided when an antigen-recognizing receptor (e.g., a CAR) binds to its target antigen. Co-stimulatory ligands include, but are not limited to, 4-1BB Ligand (4-1BBL), CD80, CD86, CD70, OX4OL, and ICOSLG. As one example, a 4-1BBL may bind to 4-1BB for providing co-stimulation signal that in combination with an activation signal induces an effector cell function of a CAR-T cell. CARs comprising an intracellular signaling domain that comprises a co-stimulatory signaling region comprising 4-1BB, ICOS or DAP-10 are disclosed in U.S. Pat. No. 7,446,190, which is herein incorporated by reference in its entirety.

In certain embodiments, the intracellular signaling domain of the CAR comprises a co-stimulatory signaling region that comprises a 4-1BB polypeptide (e.g., an intracellular domain of 4-1BB or a portion thereof). The 4-1BB polypeptide can comprise or have an amino acid sequence that is at least about 80%, at least about 85%, at least about 90%, at least about 95%, at least about 96%, at least about 97%, at least about 98%, or at least about 99%, at least about 100% homologous or identical to a sequence having a NCBI Reference No: NP_001552 (SEQ ID NO: 35) or fragments thereof, and/or may optionally comprise up to one or up to two or up to three conservative amino acid substitutions. In non-limiting certain embodiments, the 4-1BB polypeptide comprises or has an amino acid sequence that is a consecutive portion of SEQ ID NO: 35, which is at least 20, or at least 30, or at least 40, or at least 50, and up to 255 amino acids in length. Alternatively or additionally, in non-limiting various embodiments, the 4-1BB polypeptide comprises or has an amino acid sequence of amino acids 1 to 220, 1 to 50, 50 to 100, 100 to 150, 150 to 200, or 200 to 255of SEQ ID NO: 35. In certain embodiments, the intracellular signaling domain of the CAR comprises a co-stimulatory signaling region that comprises an intracellular domain of 4-1BB or a portion thereof In certain embodiments, the intracellular signaling domain of the CAR comprises a co-stimulatory signaling region that comprises an intracellular domain of human 4-1BB or a portion thereof In certain embodiments, the intracellular signaling domain of the CAR comprises a co-stimulatory signaling region that comprises a 4-1BB polypeptide comprising or having amino acids 214 to 255 of SEQ ID NO: 35. SEQ ID NO: 35 is provided below.

| [SEQ ID NO: 35] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

An exemplary nucleic acid sequence encoding amino acids 214 to 255 of SEQ ID NO: 35 is set forth in SEQ ID NO: 36, which is provided below.

[SEQ ID NO: 36]
AAACGGGGCAGAAAGAAGCTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG

In certain embodiments, the intracellular signaling domain of the CAR comprises a co-stimulatory signaling region that comprises a CD28 polypeptide (e.g., an intracellular domain of CD28 or a portion thereof). The CD28 polypeptide can comprise or have an amino acid sequence that is at least about 85%, about 90%, about 95%, about 96%, about 97%, about 98%, about 99% or 100% homologous or identical to the amino acid sequence set forth in SEQ ID NO: 29 or SEQ ID NO: 30, or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 30, which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 150 to 200, 180 to 220, or 200 to 220 of SEQ ID NO: 29 or SEQ ID NO: 30. In certain embodiments, the intracellular signaling domain of the CAR comprises a co-stimulatory signaling region that comprises the intracellular domain of CD28 or a portion thereof. In certain embodiments, the intracellular signaling domain of the CAR comprises a co-stimulatory signaling region that comprises the intracellular domain of human CD28 or a portion thereof In certain embodiments, the human CD28 has an amino acid sequence that is at least about 85%, about 90%, about 95%, about 96%, about 97%, about 98%, about 99% or 100% homologous or identical to the amino acid sequence set forth in SEQ ID NO: 30. In certain embodiments, the human CD28 has the amino acid sequence set forth in SEQ ID NO: 30. In certain embodiments, the intracellular signaling domain of the CAR comprises a co-stimulatory signaling region that comprises a CD28 polypeptide comprising or having amino acids 180 to 220 of SEQ ID NO: 30.

In certain embodiments, the intracellular signaling domain of the CAR comprises a co-stimulatory signaling region that comprises an OX40 polypeptide (e.g., the intracellular domain of OX40 or a portion thereof). The OX40 polypeptide can comprise or have an amino acid sequence that is at least about 85%, about 90%, about 95%, about 96%, about 97%, about 98%, about 99% or 100% homologous or identical to the sequence having a NCBI Reference No: NP_003318.1 (SEQ ID NO: 37), or fragments thereof, and/or may optionally comprise up to one or up to two or up to three conservative amino acid substitutions. In non-limiting certain embodiments, the OX40 polypeptide comprises or has an amino acid sequence that is a consecutive portion of SEQ ID NO: 37, which is at least 20, or at least 30, or at least 40, or at least 50, and up to 277 amino acids in length. Alternatively or additionally, in non-limiting various embodiments, the OX40 polypeptide comprises or has amino acids 1 to 220, 1 to 50, 50 to 100, 100 to 150, 150 to 200, or 200 to 277 of SEQ ID NO: 37. In certain embodiments, the intracellular signaling domain of the CAR comprises a co-stimulatory signaling region that comprises the intracellular domain of OX40 or a portion thereof. In certain embodiments, the intracellular signaling domain of the CAR comprises a co-stimulatory signaling region that comprises the intracellular domain of human OX40 or a portion thereof. In certain embodiments, the human OX40 has an amino acid sequence that is at least about 85%, about 90%, about 95%, about 96%, about 97%, about 98%, about 99% or 100% homologous or identical to the amino acid sequence set forth in SEQ ID NO: 37. In certain embodiments, the human OX40 has the amino acid sequence set forth in SEQ ID NO: 37.

SEQ ID NO: 37 is provided below:

[SEQ ID NO: 37]

| | |
|---|---|
| 1 | MCVGARRLGR GPCAALLLLG LGLSTVTGLH CVGDTYPSND |
| | RCCHECRPGN GMVSRCSRSQ |
| 61 | NTVCRPCGPG FYNDVVSSKP CKPCTWCNLR SGSERKQLCT |
| | ATQDTVCRCR AGTQPLDSYK |
| 121 | PGVDCAPCPP GHFSPGDNQA CKPWTNCTLA GKHTLQPASN |
| | SSDAICEDRD PPATQPQETQ |
| 181 | GPPARPITVQ PTEAWPRTSQ GPSTRPVEVP GGRAVAAILG |
| | LGLVLGLLGP LAILLALYLL |
| 241 | RRDQRLPPDA HKPPGGGSFR TPIQEEQADA HSTLAKI |

In certain embodiments, the intracellular signaling domain of the CAR comprises a co-stimulatory signaling region that comprises an ICOS polypeptide (e.g., the intracellular domain of ICOS or a portion thereof). The ICOS polypeptide can comprise or have an amino acid sequence that is at least about 85%, about 90%, about 95%, about 96%, about 97%, about 98%, about 99% or 100% homologous or identical to the sequence having a NCBI Reference No: NP_036224.1 (SEQ ID NO: 38), or fragments thereof, and/or may optionally comprise up to one or up to two or up to three conservative amino acid substitutions. In non-limiting certain embodiments, the ICOS polypeptide comprises or has an amino acid sequence that is a consecutive portion of SEQ ID NO: 38, which is at least 20, or at least 30, or at least 40, or at least 50, and up to 199 amino acids in length. Alternatively or additionally, in non-limiting various embodiments, the ICOS polypeptide comprises or has an amino acid sequence of amino acids 1 to 220, 1 to 50, 50 to 100, 100 to 150, or 150 to 199 of SEQ ID NO: 38. In certain embodiments, the intracellular signaling domain of the CAR comprises a co-stimulatory signaling region that comprises an intracellular domain of ICOS. In certain embodiments, the intracellular signaling domain of the CAR comprises a co-stimulatory signaling region that comprises an intracellular domain of human ICOS. In certain embodiments, the human ICOS has an amino acid sequence that is at least about 85%, about 90%, about 95%, about 96%, about 97%, about 98%, about 99% or 100% homologous or identical to the amino acid sequence set forth in SEQ ID NO: 38. In certain embodiments, the human ICOS has the amino acid sequence set forth in SEQ ID NO: 38.

SEQ ID NO: 38 is provided below:

[SEQ ID NO: 38]

| | |
|---|---|
| 1 | MKSGLWYFFL FCLRIKVLTG EINGSANYEM FIFHNGGVQI |
| | LCKYPDIVQQ FKMQLLKGGQ |
| 61 | ILCDLIKTKG SGNTVSIKSL KFCHSQLSNN SVSFFLYNLD |
| | HSHANYYFCN LSIFDPPPFK |
| 121 | VTLIGGYLHI YESQLCCQLK FWLPIGCAAF VVVCILGCIL |
| | ICWLTKKKYS SSVHDPNGEY |
| 181 | MFMRAVNTAK KSRLTDVTL |

In certain embodiments, the CAR comprises two co-stimulatory signaling domains, wherein the first co-stimulatory domain comprises an intracellular domain of 4-1BB or a portion thereof), and the second co-stimulatory domain comprises an intracellular domain of CD28 or a portion thereof).

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

### 5.3.3.4. Exemplary CARs

In certain embodiments, a presently disclosed cell comprises a CAR comprising an extracellular antigen-binding domain that binds to CD19, a transmembrane domain comprising a CD8 polypeptide (e.g., the transmembrane domain of human CD8 or a portion thereof), and an intracellular signaling domain comprising a CD3ζ polypeptide and a co-stimulatory signaling region comprising a 4-1BB polypeptide (e.g., the intracellular domain of human 4-1BB or a portion thereof).

In certain embodiments, the CAR is designated as “19BBz”. In certain embodiments, the CAR (e.g., 19BBz) comprises an extracellular antigen-binding domain comprising a V_{H} CDR1 comprising the amino acid sequence set forth in SEQ ID NO: 19, a V_{H} CDR2 comprising the amino acid sequence set forth in SEQ ID NO: 20, a V_{H} CDR3 comprising the amino acid sequence set forth in SEQ ID NO: 21, a V_{L} CDR1 comprising the amino acid sequence set forth in SEQ ID NO: 22, a V_{L} CDR2 comprising the amino acid sequence set forth in SEQ ID NO: 23, a V_{L} CDR3 comprising the amino acid sequence set forth in SEQ ID NO: 24; a transmembrane domain comprising a CD8 polypeptide that comprises the amino acid sequence set forth in SEQ ID NO: 3 or amino acids 137 to 207 of SEQ ID NO: 27; an intracellular signaling domain comprising a CD3ζ polypeptide that comprises the amino acid sequence set forth in SEQ ID NO: 33, and a co-stimulatory signaling region comprising a 4-1BB polypeptide that comprises amino acids 214 to 255 of SEQ ID NO: 35).

In certain embodiments, the CAR (e.g., 19BBz) comprises an amino acid sequence that is at least about 85%, about 90%, about 95%, about 96%, about 97%, about 98%, about 99% or about 100% homologous or identical to the amino acid sequence set forth in SEQ ID NO: 39, which is provided below.

[SEQ ID NO: 39]
EVKLQQSGAELVRPGSSVKISCKASGYAFSSYWMNWVKQRPGQGLEWIGQ
IYPGDGDTNYNGKFKGQATLTADKSSSTAYMQLSGLTSEDSAVYFCARKT
ISSVVDFYFDYWGQGTTVTVSSGGGGSGGGGSGGGGSDIELTQSPKFMST
SVGDRVSVTCKASQNVGTNVAWYQQKPGQSPKPLIYSATYRNSGVPDRFT
GSGSGTDFTLTITNVQSKDLADYFCQQYNRYPYTSGGGTKLEIKRAAAPT
TTPAPRPPTPAPTIASQPLSLRPEACRPAAGGAVHTRGLDFACDIYIWAP
LAGTCGVLLLSLVITLYCNKRGRKKLLYIFKQPFMRPVQTTQEEDGCSCR
FPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGL
YQGLSTATKDTYDALHMQALPPR

An exemplary nucleic acid sequence encoding the amino acid sequence of SEQ ID NO: 39 is set forth in SEQ ID NO: 40, which is provided below.

[SEQ ID NO: 40]
GAGGTGAAGCTGCAGCAGTCTGGGGCTGAGCTGGTGAGGCCTGGGTCCTC
AGTGAAGATTTCCTGCAAGGCTTCTGGCTATGCATTCAGTAGCTACTGGA
TGAACTGGGTGAAGCAGAGGCCTGGACAGGGTCTTGAGTGGATTGGACAG
ATTTATCCTGGAGATGGTGATACTAACTACAATGGAAAGTTCAAGGGTCA
AGCCACACTGACTGCAGACAAATCCTCCAGCACAGCCTACATGCAGCTCA
GCGGCCTAACATCTGAGGACTCTGCGGTCTATTTCTGTGCAAGAAAGACC
ATTAGTTCGGTAGTAGATTTCTACTTTGACTACTGGGGCCAAGGGACCAC
GGTCACCGTCTCCTCAGGTGGAGGTGGATCAGGTGGAGGTGGATCTGGTG
GAGGTGGATCTGACATTGAGCTCACCCAGTCTCCAAAATTCATGTCCACA
TCAGTAGGAGACAGGGTCAGCGTCACCTGCAAGGCCAGTCAGAATGTGGG
TACTAATGTAGCCTGGTATCAACAGAAACCAGGACAATCTCCTAAACCAC
TGATTTACTCGGCAACCTACCGGAACAGTGGAGTCCCTGATCGCTTCACA
GGCAGTGGATCTGGGACAGATTTCACTCTCACCATCACTAACGTGCAGTC
TAAAGACTTGGCAGACTATTTCTGTCAACAATATAACAGGTATCCGTACA
CGTCCGGAGGGGGGACCAAGCTGGAGATCAAACGGGCGGCCGCACCCACC
ACGACGCCAGCGCCGCGACCACCAACCCCGGCGCCCACGATCGCGTCGCA
GCCCCTGTCCCTGCGCCCAGAGGCGTGCCGGCCAGCGGCGGGGGGCGCAG
TGCACACGAGGGGGCTGGACTTCGCCTGTGATATCTACATCTGGGCGCCC
CTGGCCGGGACTTGTGGGGTCCTTCTCCTGTCACTGGTTATCACCCTTTA
CTGCAACAAACGGGGCAGAAAGAAGCTCCTGTATATATTCAAACAACCAT
TTATGAGACCAGTACAAACTACTCAAGAGGAAGATGGCTGTAGCTGCCGA
TTTCCAGAAGAAGAAGAAGGAGGATGTGAACTGAGAGTGAAGTTCAGCAG
GAGCGCAGACGCCCCCGCGTACCAGCAGGGCCAGAACCAGCTCTATAACG
AGCTCAATCTAGGACGAAGAGAGGAGTACGATGTTTTGGACAAGAGACGT
GGCCGGGACCCTGAGATGGGGGGAAAGCCGAGAAGGAAGAACCCTCAGGA
AGGCCTGTACAATGAACTGCAGAAAGATAAGATGGCGGAGGCCTACAGTG
AGATTGGGATGAAAGGCGAGCGCCGGAGGGGCAAGGGGCACGATGGCCTT
TACCAGGGTCTCAGTACAGCCACCAAGGACACCTACGACGCCCTTCACAT
GCAGGCCCTGCCCCCTCGC

In certain embodiments, the CAR (e.g., 19BBz) further comprises a CD8 signal peptide. In certain embodiments, the CD8 signal peptide comprises or has the amino acid sequence set forth in SEQ ID NO: 10.An exemplary nucleic acid sequence encoding the amino acid sequence of SEQ ID NO: 10 is set forth in SEQ ID NO: 41, which is provided below.

[SEQ ID NO: 41]
ATGGCTCTCCCAGTGACTGCCCTACTGCTTCCCCTAGCGCTTCTCCTGCA
TGCA

The amino acid sequence for the 19BBz comprising the CD8 signal peptide is set forth in

SEQ ID NO: 42, which is provided below.

[SEQ ID NO: 42]
MALPVTALLLPLALLLHAEVKLQQSGAELVRPGSSVKISCKASGYAFSSY
WMNWVKQRPGQGLEWIGQIYPGDGDTNYNGKFKGQATLTADKSSSTAYMQ
LSGLTSEDSAVYFCARKTISSVVDFYFDYWGQGTTVTVSSGGGGSGGGGS
GGGGSDIELTQSPKFMSTSVGDRVSVTCKASQNVGTNVAWYQQKPGQSPK
PLIYSATYRNSGVPDRFTGSGSGTDFTLTITNVQSKDLADYFCQQYNRYP
YTSGGGTKLEIKRAAAPTTTPAPRPPTPAPTIASQPLSLRPEACRPAAGG
AVHTRGLDFACDIYIWAPLAGTCGVLLLSLVITLYCNKRGRKKLLYIFKQ
PFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAY
SEIGMKGERRRGKGHDGLYQGLSTATKDTYDALHMQALPPR

An exemplary nucleic acid sequence encoding the amino acid sequence of SEQ ID NO: 42 is set forth in SEQ ID NO: 43, which is provided below.

[SEQ ID NO: 43]
ATGGCTCTCCCAGTGACTGCCCTACTGCTTCCCCTAGCGCTTCTCCTGCA
TGCAGAGGTGAAGCTGCAGCAGTCTGGGGCTGAGCTGGTGAGGCCTGGGT
CCTCAGTGAAGATTTCCTGCAAGGCTTCTGGCTATGCATTCAGTAGCTAC
TGGATGAACTGGGTGAAGCAGAGGCCTGGACAGGGTCTTGAGTGGATTGG
ACAGATTTATCCTGGAGATGGTGATACTAACTACAATGGAAAGTTCAAGG
GTCAAGCCACACTGACTGCAGACAAATCCTCCAGCACAGCCTACATGCAG
CTCAGCGGCCTAACATCTGAGGACTCTGCGGTCTATTTCTGTGCAAGAAA
GACCATTAGTTCGGTAGTAGATTTCTACTTTGACTACTGGGGCCAAGGGA
CCACGGTCACCGTCTCCTCAGGTGGAGGTGGATCAGGTGGAGGTGGATCT
GGTGGAGGTGGATCTGACATTGAGCTCACCCAGTCTCCAAAATTCATGTC
CACATCAGTAGGAGACAGGGTCAGCGTCACCTGCAAGGCCAGTCAGAATG
TGGGTACTAATGTAGCCTGGTATCAACAGAAACCAGGACAATCTCCTAAA
CCACTGATTTACTCGGCAACCTACCGGAACAGTGGAGTCCCTGATCGCTT
CACAGGCAGTGGATCTGGGACAGATTTCACTCTCACCATCACTAACGTGC
AGTCTAAAGACTTGGCAGACTATTTCTGTCAACAATATAACAGGTATCCG
TACACGTCCGGAGGGGGGACCAAGCTGGAGATCAAACGGGCGGCCGCACC
CACCACGACGCCAGCGCCGCGACCACCAACCCCGGCGCCCACGATCGCGT
CGCAGCCCCTGTCCCTGCGCCCAGAGGCGTGCCGGCCAGCGGCGGGGGGC
GCAGTGCACACGAGGGGGCTGGACTTCGCCTGTGATATCTACATCTGGGC
GCCCCTGGCCGGGACTTGTGGGGTCCTTCTCCTGTCACTGGTTATCACCC
TTTACTGCAACAAACGGGGCAGAAAGAAGCTCCTGTATATATTCAAACAA
CCATTTATGAGACCAGTACAAACTACTCAAGAGGAAGATGGCTGTAGCTG
CCGATTTCCAGAAGAAGAAGAAGGAGGATGTGAACTGAGAGTGAAGTTCA
GCAGGAGCGCAGACGCCCCCGCGTACCAGCAGGGCCAGAACCAGCTCTAT
AACGAGCTCAATCTAGGACGAAGAGAGGAGTACGATGTTTTGGACAAGAG
ACGTGGCCGGGACCCTGAGATGGGGGGAAAGCCGAGAAGGAAGAACCCTC
AGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGG
CCTTTACCAGGGTCTCAGTACAGCCACCAAGGACACCTACGACGCCCTTC
ACATGCAGGCCCTGCCCCCTCGC

### 5.4. Cells

The presently disclosed subject matter provides cells comprising (a) a ligand-recognizing receptor (e.g., a ligand-recognzing receptor disclosed in Section 5.3), and (b) an IgG-degrading enzyme (e.g., an IgG-degrading enzyme disclosed in Section 5.2). In certain embodiments, the ligand-recognizing receptor is capable of activating the cell. The cells can be transduced with the ligand-recognizing receptor and the IgG-degrading enzyme such that the cells co-express the ligand-recognizing receptor and the IgG-degrading enzyme. In certain embodiments, the IgG-degrading enzyme is attached to the cell surface. In certain embodiments, the IgG-degrading enzyme is not attached to the cell surface, and is delivered or released from the cells.

In certain embodiments, the cell further comprises a cleavable (e.g., self-cleavable) linker (e.g., a 2A peptide, e.g., a P2A peptide, a T2A peptide, an E2A peptide, and a F2A peptide). In certain embodiments, the cell further comprises a P2A peptide. In certain embodiments, the P2A peptide is positioned between the ligand-recognizing receptor and the IgG-degrading enzyme. In certain embodiments, the P2A peptide comprises or has the amino acid sequence set forth in SEQ ID NO: 44, which is provided below:

[SEQ ID NO: 44]
ATNFSLLKQAGDVEENPGP

An exemplary nucleotide sequence encoding the amino acid sequence set forth in SEQ ID NO: 44 is set forth in SEQ ID NO: 45, which is provided below.

[SEQ ID NO: 45]
GCTACTAACTTCAGCCTGCTGAAGCAGGCTGGAGACGTGGAGGAGAACCC
TGGACCT

In certain embodiments, the cell is a responsive cell. In certain embodiments, the cell is a responsive cell, e.g., an immunoresponsive cell. In certain embodiments, the cell is a activatable cell. In certain embodiments, the cell is a cell of the lymphoid lineage. In certain embodiments, the cell is a cell of the myeloid lineage. In certain embodiments, the cell is a cell from a normal tissue, e.g., from kidney, liver, lung, bone marrow, or pancreas.

Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, and stem cells from which lymphoid cells may be differentiated. The stem cells can be pluripotent stem cells (e.g., embryonic stem cells, and induced pluripotent stem cells).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), tumor-infiltrating lymphocyte (TIL), Natural Killer T cells (NK T cells), Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural Killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M.C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells. In certain embodiments, the cell is an allogeneic cell.

In certain embodiments, the cell is a cell of the myeloid lineage. Non-limiting examples of cells of the myeloid lineage include monocytes, macrophages, neutrophils, basophils, eosinophils, erythrocytes, megakaryocytes, and stem cells from which myeloid cells may be differentiated.

In certain embodiments, the presently disclosed cells are used in a therapy. In certain embodiments, the presently disclosed cells are used in a cell therapy. In certain embodiments, the presently disclosed cells are used in a genetic therapy. In certain embodiments, the presently disclosed cells are used in a CRISPR gene therapy. The field of cell engineering is expanding, especially as the use of CRISPR-Cas9 technology is becoming widespread, and with the introduction of multiple foreign proteins into cells, immunogenicity of the cells becomes an important concern. Immunogenic cells would be rapidly eliminated from the patient, reducing their effectiveness (Porter et al., Science Translational Medicine (2015); 7; Maude et al., N Engl J Med. (2014); 371:1507-1517; Louis et al., Blood (2011); 118:6050-6056). Gene therapies involve inserting foreign genes into cells often accompanied by viral genes or other foreign helper genes. The viral proteins, such as from AAV virus used in hemophilia treatment and other genetic disorders, may persist in the patient for months or years and serve as a target of the immune response.

The presently disclosed cells can mitigate the immunogenicity of foreign cells.

In certain embodiments, the presently disclosed cells are used in an immunotherapy. In certain embodiments, the presently disclosed cells are used in an adoptive cell transfer (ACT). A rapid emerging and personalized type of immunotherapy is adoptive cell transfer (ACT), where patients' immune cells are used as the tool to treat their cancer (Kalos et al., Immunity (2013); 39:49-60). T-cells can be genetically engineered to recognize tumor cells, expanded in vitro, and then transferred back into the patients. There are several types of ACT including chimeric antigen receptor (CAR) T-cells, T-cell receptor (TCR)-engineered T-cells, and tumor infiltrating lymphocytes (TILs) (Rosenberg et al., Nature Reviews Cancer (2008); 8:299-308). In ACT, T-cells are genetically engineered to recognize tumor cells, expanded in vitro, and then transferred back into the patients.

CAR T-cell therapy has made progress in the clinical setting with two FDA approved therapies in 2017 (Zheng et al., Drug Discovery Today (2018); 23:1175-1182). Continued efforts in the field are addressing the current limitations of CAR T-cell therapies to improve trafficking and recognition of the tumor, increase their proliferation and persistence, and enhance our control over their activity (Lim et al., Cell (2017); 168:724-740). There are needs for improved ACTs with reduced off-target effects and decreased toxicity, as well as improved the overall efficacy. Humoral responses in patients have been observed to CAR T-cells in patients receiving them. Such antibodies were directed towards the CAR construct proteins, as well as against proviral proteins from the retroviral vector used for transduction (Kershaw et al., Clinical Cancer Research (2006); 12:6106-6115; Lamers et al., Blood (2011); 117:72-82; Jensen et al., Biology of Blood and Marrow Transplantation (2010); 16:1245-1256.7-9). Immunogenicity may also become a more prevalent issue as bacterial proteins are used for CAR T-cell engineering through the use of CRISPR technology, and importantly, as the use of allogeneic CAR T-cells becomes more widespread (Jung et al., Molecules and Cells (2018); 41:717-723; Graham et al., Cells (2018); 7:155).

The presently disclosed cells can improve the ACT efficacy, and/or reduce toxicity in the context of antigenicity to CAR T-cells.

The presently disclosed cells have increased resistance to a humoral response, which allows for prolonged peripheral persistence of the CAR T-cells, thereby leading to more potent activities (e.g., anti-tumor activities). The prolonged persistence of the cells can also improve the cost effectiveness of the cell therapy (e.g., ACT, which is usually associated with very high costs).

Antibody binding to CAR-T cells can lead to the lysis of CAR T-cells by antibody-dependent cell-mediated cytotoxicity (ADCC) or complement-dependent cytotoxicity (CDC) thus yielding a decreased therapeutic effect. Anti-idiotype antibodies have been shown to neutralize CAR T-cell function (Lamers et al., Blood (2011); 117:72-82). Limited peripheral persistence of CAR T-cells has also been attributed to cellular responses, where CAR T-cells are targeted by endogenous T cells (Lamers et al., Blood (2011); 117:72-82; Jensen et al., Biology of Blood and Marrow Transplantation (2010); 16:1245-1256). The epitopes responsible for anti-CAR immunity observed in a carbonic anhydrase IX (CAIX)-targeting CAR T-cell model were identified, which included peptide sequences derived from the complementarity-determining region and the framework region of the CAR, and also proviral sequences derived from the SFG retroviral vector Lamers et al., Blood (2011); 117:72-82). One approach that has been taken on to address this issue is humanizing CARs in order to render them non-immunogenic (Gonzales et al., Tumor Biology (2005); 26:31-43). However, that would not address viral vector-specific immune responses, nor allogeneic cells. The presently disclosed cells can overcome the humoral response to foreign cellular therapies such as CAR T-cell therapies, and can prevent neutralization of cellular activity by anti-cell antibodies.

There is precedence for the formation of anti-CAR antibodies in the literature, however, there are significant hurdles to the success of these therapies (Kershaw et al., Clinical Cancer Research (2006); 12:6106-6115; Lamers et al., Blood (2011); 117:72-82; Jensen et al., Biology of Blood and Marrow Transplantation (2010); 16:1245-1256; Jung et al., Molecules and Cells (2018); 41:717-723; Graham et al., Cells (2018); 7:155).

The presently discloses cells comprising an IgG-degrading enzyme (e.g., IdeS), which serves as a biomolecular shield against the host humoral response (e.g., potential antibodies). The expression of the IgG-degrading enzyme in the cells yields: a) protection from neutralizing anti-CAR antibodies, b) prolonged persistence of the cells (e.g., engineered CAR T-cells), and c) prolonged window of therapeutic activity, thereby yielding overall higher efficacy. Other approaches in the CAR-T cell field are focused on cellular immune responses to CAR T-cells, for example by deleting HLA I and the TCR (Zhao et al., Journal of Hematology and Oncology (2018); 11:1-9). However, to the inventors' knowledge, this is the first work that directly aims to address an antibody-driven host immune response.

### 5.5. Compositions and Vectors

The present discloses subject matter provides compositions comprising an IgG-degrading enzyme disclosed herein (e.g., disclosed in Section 5.2) and an ligand-recognizing receptor disclosed herein (e.g., disclosed in Section 5.3). Also provided are cells comprising such compositions.

In certain embodiments, the IgG-degrading enzyme is operably linked to a first promoter. In certain embodiments, the ligand-recognizing receptor is operably linked to a second promoter.

In certain embodiments, the composition further comprises a cleavable (e.g., self-cleavable) linker (e.g., a 2A peptide, e.g., a P2A peptide, a T2A peptide, an E2A peptide, and a F2A peptide). In certain embodiments, the composition further comprises a P2A peptide. In certain embodiments, the P2A peptide is positioned between the ligand-recognizing receptor and the IgG-degrading enzyme. In certain embodiments, the P2A peptide comprises or has the amino acid sequence set forth in SEQ ID NO: 43.

Furthermore, the present discloses subject matter provides nuclei acid compositions comprising a first polynucleotide encoding an IgG-degrading enzyme disclosed herein (e.g., disclosed in Section 5.2) and a second polynucleotide encoding a ligand-recognizing receptor disclosed herein (e.g., disclosed in Section 5.3). Also provided are cells comprising such nucleic acid compositions.

In certain embodiments, the nucleic acid composition further comprises a first promoter that is operably linked to the IgG-degrading enzyme. In certain embodiments, the nucleic acid composition further comprises a second promoter that is operably linked to the ligand-recognizing receptor.

In certain embodiments, one or both of the first and second promoters are endogenous or exogenous. In certain embodiments, the exogenous promoter is selected from an elongation factor (EF)-1 promoter, CMV promoter, a SV40 promoter, a PGK promoter, and a metallothionein promoter.

In certain embodiments, the nucleic acid composition further comprises a cleavable (e.g., self-cleavable) linker (e.g., a 2A peptide, e.g., a P2A peptide, a T2A peptide, an E2A peptide, and a F2A peptide). In certain embodiments, the nucleic acid composition further comprises a P2A peptide. In certain embodiments, the P2A peptide is positioned between the ligand-recognizing receptor and the IgG-degrading enzyme. In certain embodiments, the P2A peptide comprises or has the nucleotide sequence set forth in SEQ ID NO: 45.The compositions and nucleic acid compositions can be administered to subjects and/or delivered into cells by art-known methods or as described herein.

Genetic modification of a cell (e.g., an immunoresponsive cell, e.g., a T cell or a NK cell) can be accomplished by transducing a substantially homogeneous cell composition with a recombinant DNA construct. In certain embodiments, a retroviral vector (either gamma-retroviral or lentiviral) is employed for the introduction of the nucleic acid compositions into the cell. For example, a the first polynucleotide encoding the IgG-degrading enzyme and the second polynucleotide encoding the ligand-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include a ligand-recognizing receptor (e.g., a CAR or a TCR), a retroviral vector is generally employed for transduction, however any other suitable viral vector or non-viral delivery system can be used. The ligand-recognizing receptor and the IgG-degrading enzyme can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen,

Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver the IgG-degrading enzyme and/or the ligand-recognizing receptor disclosed herein to a cell or a subject. In certain embodiments, a CRISPR system is used to deliver the IgG-degrading enzyme and/or the ligand-recognizing receptor disclosed herein. In certain embodiments, zinc-finger nucleases are used to deliver the IgG-degrading enzyme and/or the ligand-recognizing receptor disclosed herein. In certain embodiments, a TALEN system is used to deliver the IgG-degrading enzyme and/or the ligand-recognizing receptor disclosed herein.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease Fokl. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor la enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

Methods for delivering the genome editing agents/systems can vary depending on the need. In certain embodiments, the components of a selected genome editing method are delivered as nucleic acid compositions (e.g., DNA constructs) in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

The composition or nucleic acid composition disclosed herein can be placed anywhere in a genome. In certain embodiments, the composition or nucleic acid composition is placed in a site within the genome of a T cell.

### 5.6. Polypeptides and Analogs

Also included in the presently disclosed subject matter are polypeptides disclosed herein (e.g., CD19, 4-1BB, CD28, CD3ζ, and IgG-degrading enzyme or fragments thereof) that are modified in ways for desired purpose, e.g., for enhancing their anti-neoplastic and/or anti-tumor activity when expressed in a cell. The presently disclosed subject matter provides methods for optimizing an amino acid sequence or nucleic acid sequence by producing an alteration in the sequence, and modified amino acid sequences and nucleic acid sequences. Such alterations may include certain mutations, deletions, insertions, or post-translational modifications. The presently disclosed subject matter further includes analogs of any naturally-occurring polypeptide disclosed herein. Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amina acids, e.g., D-amino acids or non-naturally occurring or synthetic amino acids, e.g., β or γ amino acids.

In addition to full-length polypeptides, the presently disclosed subject matter also provides fragments of any one of the polypeptides disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein disclosed herein (e.g., an IgG-degrading enzyme). Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in a cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### 5.7. Administration

Compositions comprising the presently disclosed cells can be provided systemically or directly to a subject for inducing and/or enhancing an immune response to an antigen and/or treating and/or preventing a neoplasia, pathogen infection, or infectious disease. In certain embodiments, the presently disclosed cells, compositions, or nucleic acid compositions f are directly injected into an organ of interest (e.g., an organ affected by a neoplasm). Alternatively, the presently disclosed cells, compositions, or nucleic acid compositions are provided indirectly to the organ of interest, for example, by administration into the circulatory system (e.g., the tumor vasculature). Expansion and differentiation agents can be provided prior to, during or after administration of the cells, compositions, or nucleic acid compositions to increase production of the cells (e.g., T cells (e.g., CTL cells) or NK cells in vitro or in vivo.

The presently disclosed cells, compositions, or nucleic acid compositions can be administered in any suitable routes, including but not limited to, intravenous, subcutaneous, intranodal, intratumoral, intrathecal, intrapleural, intraperitoneal, and cutaneous. In certain embodiments, the presently disclosed cells, compositions, or nucleic acid compositions are administrated intraperitoneally to a subject. Usually, at least about 1×10⁵ cells will be administered, eventually reaching about 1×10¹⁰ or more. The presently disclosed cells can comprise an impure or purified population of cells. Those skilled in the art can readily determine the percentage of the presently disclosed cells in a population using various well-known methods, such as fluorescence activated cell sorting (FACS). Suitable ranges of purity in populations comprising the presently disclosed cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like. The cells may be comprised of an organ or tissue, including 10⁹ or up to 10¹¹ cells, of various lineages.

The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed immunoresponsive cell), it can be formulated in a unit dosage injectable form (solution, suspension, emulsion).

### 5.8. Formulations

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In certain embodiments, between about 10⁴ and about 10¹⁰, between about 10⁵ and about 10⁹, or between about 10⁶ and about 10⁸ of the presently disclosed immunoresponsive cells are administered to a subject (e.g., a human subject). In certain embodiments, between about 10⁴ and about 10⁷, or between about 10⁵ and about 10⁷ of the presently disclosed immunoresponsive cells are administered to a subject (e.g., a human subject). More effective cells may be administered in even smaller numbers. In certain embodiments, at least about 1×10⁸, about 2×10⁸, about 3×10⁸, about 4×10⁸, or about 5×10⁸ of the presently disclosed immunoresponsive cells are administered to a subject (e.g., a human subject). The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt% or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### 5.9. Methods of Uses

The presently disclosed subject matter provides methods for administering a presently disclosed cell, a presently disclosed composition, or a presently disclosed nucleic acid composition into a subject, e.g., for a treatment or therapy. Non-limiting examples of treatments or therapies include immunotherapies (e.g., adoptive cell transfer), cell therapies (or cellular therapies), stem cell transplants, organ transplants, genetic therapies (e.g., CRISPR gene editing therapies), virus infusions (e.g., AAV), nanoparticles containing nucleic acids, free nucleic acids or analogs that are rendered more stable, mRNA or stabilized mRNA, or organs or tissues containing the subject engineered cells. The presently disclosed cells, compositions, and nucleic acid compositions can be used in a therapy, a treatment, or a medicament. In certain embodiments, increased resistance to host humoral responses, prolonged persistence of cells, and/or mitigated immunogenicity of foreign cells are desired for the therapy or treatment.

The presently disclosed subject matter provides methods for treating and/or preventing a neoplasia in a subject. The presently disclosed cells, compositions, and nucleic acid compositions can be used for treating and/or preventing a neoplasia in a subject. The presently disclosed cells, compositions, and nucleic acid compositions can be used for prolonging the survival of a subject suffering from a neoplasia.

The presently disclosed subject matter provides methods for treating and/or preventing a pathogen infection or other infectious disease in a subject, such as an immunocompromised human subject. The presently disclosed cells, compositions, and nucleic acid compositions can also be used for treating and/or preventing a pathogen infection or other infectious disease in a subject, such as an immunocompromised human subject. Such methods comprise administering the presently disclosed cells in an amount effective, a presently disclosed composition (e.g., a pharmaceutical composition), or a presently disclosed nucleic acid composition to achieve the desired effect, be it palliation of an existing condition or prevention of recurrence.

The presently disclosed subject matter provides methods for treating and/or preventing an autoimmune disease in a subject. The presently disclosed cells, compositions, and nucleic acid compositions can also be used for treating and/or preventing an autoimmune disease in a subject. Such methods comprise administering the presently disclosed cells in an amount effective, a presently disclosed composition (e.g., a pharmaceutical composition), or a presently disclosed nucleic acid composition to a subject having an autoimmune disease.

The presently disclosed subject matter provides methods for reducing and/or preventing an antibody-mediated rejection of cells and/or tissues in a subject, wherein the subject receives an organ transplant. The presently disclosed cells, compositions, and nucleic acid compositions can also be used for reducing and/or preventing an antibody-mediated rejection of cells and/or tissues in a subject, wherein the subject receives an organ transplant. Such methods comprise administering the presently disclosed cells in an amount effective, a presently disclosed composition (e.g., a pharmaceutical composition), or a presently disclosed nucleic acid composition to a subject who receives an organ transplant.

The presently disclosed subject matter provides methods for reducing and/or preventing an antibody-mediated rejection of autologous or allogeneic cells and/or tissues in a subject, wherein the subject receives a cell therapy. The presently disclosed cells, compositions, and nucleic acid compositions can also be used for reducing and/or preventing an antibody-mediated rejection of cells and/or tissues, wherein the subject receives a cell therapy. Such methods comprise administering the presently disclosed cells in an amount effective, a presently disclosed composition (e.g., a pharmaceutical composition), or a presently disclosed nucleic acid composition to a subject who receives a cell therapy.

For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

For adoptive immunotherapy using antigen-specific T cells, cell doses in the range of about 10⁶-10¹⁰ (e.g., about 10⁹) are typically infused. Upon administration of the presently disclosed cells into the host and subsequent differentiation, T cells are induced that are specifically directed against the specific antigen.

### Neoplasia

The presently disclosed subject matter provides methods for treating and/or preventing a neoplasia in a subject. The method can comprise administering an effective amount of the presently disclosed cells, a presently disclosed composition, or a presently disclosed nucleic acid composition to a subject having a neoplasia.

Non-limiting examples of neoplasia include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasia is selected from blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed cells, compositions, nucleic acid compositions can be used for treating and/or preventing blood cancers (e.g., leukemias, lymphomas, and myelomas) or ovarian cancer, which are not amenable to conventional therapeutic interventions. In certain embodiments, the presently disclosed cells, compositions, nucleic acid compositions can be used for treating and/or preventing a solid tumor. In certain embodiments, the presently disclosed cells, compositions, nucleic acid compositions can be used for treating and/or preventing a neoplasia selected from acute myeloid leukemia (AML), lymphoblastic leukemia (ALL), chronic lymphocytic leukemia (CLL), chronic myeloid leukemia (CIVIL), multiple myeloma, non-Hodgkin's lymphoma, Hodgkin's lymphoma breast cancer, ovarian cancer, mesothelioma, gliobastoma, colorectal cancer, and pancreas cancer.

### Autoimmune Diseases

The presently disclosed subject matter provides methods for treating and/or preventing an autoimmune disease in a subject. The method can comprise administering an effective amount of the presently disclosed cells, a presently disclosed composition, or a presently disclosed nucleic acid composition to a subject having an autoimmune disease. Non-limiting examples of autoimmune diseases include rheumatoid arthritis, myasthenia gravis, systemic lupus, Graves disease, Hashimoto's thyroiditis, systemic sclerosis, biliary cirrhosis, celiac disease, axonal neuropathy, inflammatory myopathy, cerebellar degenerations, diabetes mellitus type 1, and polymyositis.

There are more than 20 million patients with autoimmune disorders in the USA. Many of these, such as Lupus and myesthenia gravis involve attack by antibodies on the patient's own tissue components, DNA and cells. https://www.google.com/search?client=firefox-b-1-d&q=incidence+outimmune+disease. There are few effective nor curable approaches to these diseases. IgG plays an important protective role in the human immune system, but is also associated in the pathogenesis of diseases such as rheumatoid arthritis, myasthenia gravis, systemic lupus etc., where removal of IgG has been used as a therapeutic avenue to treat these autoimmune diseases (Johansson et al., PLoS ONE (2008); 3:1-6; Berta et al., The International Journal of Artificial Organs (1994); 17:603-608; Stummvoll et al., Annals of the Rheumatic Diseases (2005); 64:1015-1021). The IgG-degrading enzyme comprised in the presently disclosed cells can deplete the functional IgG attacking the host cells, thereby treating an autoimmune disease.

### Antibody-Mediated Rejections

The presently disclosed subject matter provides methods for reducing and/or preventing an antibody-mediated rejection of cells and/or tissues in a subject. In certain embodiments, the subject receives an organ transplant. In certain embodiments, the transplant is an allogeneic transplant (allotransplant). In certain embodiments, the subject receives the presently disclosed cells, composition, or nucleic acid composition prior to the organ transplant. In certain embodiments, the subject receives a cell therapy, e.g., the cells and/or tissues (e.g., autologous or allogeneic cells and/or tissues) are used in the cell therapy.

The method can comprise administering an effective amount of the presently disclosed cells, a presently disclosed composition, or a presently disclosed nucleic acid composition to the subject.

Solid organ transplants such as for kidney, liver, lung, heart and other organs are used in more than 36,000 patients per year in the USA and >100,000 are waiting for transplants. https://imw.organdonor.gov/statistics-stories/statistics.html. These organs are matched as well as possible to the patient but immunosuppression of the patient with serious and sometimes fatal consequences, is frequent and life-long. The cost in the USA is $100,000,000,000. Host IgG plays an important role in allotransplants, where incompatibility between HLA donors leads to antibody-mediated rejection of allografts (Loupy et al., New England Journal of Medicine (2018); 379:1150-1160). IdeS was evaluated in humans for desensitization prior to allotransplants. In the study, 24 out of 25 patients were able to receive HLA-incompatible transplants, after treatment with IdeS which rapidly removed all donor-specific antibodies (Jordan et al., New England Journal of Medicine (2017); 377:442-453; Lonze et al., Annals of Surgery (2018); 268:488-496). _The IgG-degrading enzyme comprised in the presently disclosed cells can deplete the functional IgG (e.g., host IgG), attacking the donor organ cells, thereby reducing and/or preventing antibody-mediated rejection associated with an organ transplant.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of neoplasm, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes.

Another group have a genetic predisposition to neoplasia but have not yet evidenced clinical signs of neoplasia. For instance, women testing positive for a genetic mutation associated with breast cancer, but still of childbearing age, can wish to receive one or more of the cells described herein in treatment prophylactically to prevent the occurrence of neoplasia until it is suitable to perform preventive surgery.

As a consequence of expression of a ligand-recognizing receptor (e.g., an antigen-recognizing receptor that binds to a tumor antigen) and an IgG-degrading enzyme that enhances the activity of the cell (e.g., anti-tumor activities), adoptively transferred cells are endowed with augmented and selective cytolytic activity at the tumor site. Furthermore, subsequent to their localization to tumor or viral infection and their proliferation, the cells (e.g., T cells) turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed cells, a presently disclosed composition, or a presently disclosed nucleic acid composition to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed immunoresponsive cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the antigen-recognizing receptor of a presently disclosed CAR. The suicide gene can be included within the vector comprising nucleic acids encoding a presently disclosed CAR. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated CAR-expressing T cells. The incorporation of a suicide gene into the a presently disclosed CAR gives an added level of safety with the ability to eliminate the majority of CAR T cells within a very short time period. A presently disclosed cell (e.g., a T cell) incorporated with a suicide gene can be pre-emptively eliminated at a given timepoint post CAR T cell infusion, or eradicated at the earliest signs of toxicity.

### 5.10. Kits

The presently disclosed subject matter provides kits for treating and/or preventing a neoplasia, or a pathogen infection, or an autoimmune disease in a subject, and kits for reducing and/or preventing antibody-mediated rejection in a subject, wherein the subject receives an organ transplant. In certain embodiments, the kit comprises an effective amount of presently disclosed cells, a presently disclosed composition, or a presently disclosed nucleic acid composition. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an antigen-recognizing receptor (e.g., a CAR or a TCR) directed toward an antigen of interest and an isolated nucleic acid molecule encoding an IL-36 polypeptide in expressible (and secretable) form, which may optionally be comprised in the same or different vectors.

If desired, the cells, composition, or nucleic acid composition are provided together with instructions for administering the cells, composition, or nucleic acid composition to a subject having or at risk of developing a neoplasia, pathogen infection, or an autoimmune disease or a subject receiving an organ transplant. The instructions generally include information about the use of the cells, the composition or nucleic acid composition for the treatment and/or prevention of a neoplasia or a pathogen infection, or autoimmune disease. In certain embodiments, the instructions include at least one of the following: description of the therapeutic agent; dosage schedule and administration for treatment or prevention of a neoplasm, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1

### Generation and In Vitro Activities

### Summary

Two forms of CAR T-cells expressing IdeS were generated, i.e., membrane-bound form (the IdeS was expressed as a surface membrane-bound form) and secreted form (the IdeS was secreted from the cells). As shown below, in both form, IdeS successfully cleaved IgG in vitro.

### Results

### Constructs and Production of Stable Lines

CAR T-cell therapies targeting CD19, an antigen of B-cell tumors, were recently approved by the FDA (Zheng et al., Drug Discovery Today (2018); 23:1175-1182). CD19 makes for an ideal antigen as it is specific to B-cells and it is normally not expressed on other cells or tissues. The CD19 CAR with the 4-1BB/CD3ζ signaling domain has been well characterized in vitro and in vivo in patients (Brentjens et al., Sci Transl Med. (2013); 5:177ra38; Pegram et al., Blood (2012); 119:4133-4141; Kalos et al., Science Translational Medicine (2011); 3:1-11).

As shown in FIG. 1, a membrane-bound form construct (referred to as “IdeS-tm”; see FIG. 1A) and a secreted form construct (referred to as “IdeS-sec”; see FIG. 1B) were generated. The T cells were engineered to express a CD19-targeted CAR comprising an intracellular signaling domain that comprises a 4-1BB polypeptide and a CD3ζ polypeptide), and a transmembrane domain that comprises a CD8 polypeptide. The CAR is designated as “19BBz”.

The design of the constructs was based on previously reported methods using the SFG gamma retroviral vector (Rivière et al., Proceedings of the National Academy of Sciences of the United States of America (1995); 92:6733-7). The CD8 signal peptide sequence was used to transport IdeS to the cell membrane (see FIGS. 1A and 1B). To generate a membrane-bound version of the enzyme, the transmembrane domain of CD8 was incorporated on the C-terminus of the enzyme. The CAR construct was then added after the self-cleaving peptide 2A. As shown in FIGS. 1A and 1B, the CAR construct includes a CD8 signal peptide sequence, the antigen-specific scFv (in this case anti-CD19), a CD8 transmembrane domain, and an 4-1BB/CD3 intracellular signaling domain. The process to produce CAR expressing T-cells was previously described in Brentjens et al., Sci Transl Med. (2013); 5:177ra38; Parente-Pereira et al., Journal of Biological Methods (2014); 1:7). The H29 retro-viral packaging cell line was transfected using the constructs. Virus derived from H29 cells was used to produce stable PG13 retroviral packaging cell lines. PG13 cells produced gibbon ape leukemia virus (GALV) particles, which were used to transduce any cell lines, or primary cells (Parente-Pereira et al., Journal of Biological Methods (2014); 1:7). Peripheral blood mononuclear cells (PBMCs) were transduced to generate the CAR T-cells. In addition, Galv9 producer cell line was used to produce virus.

### Expression and In vitro Activity

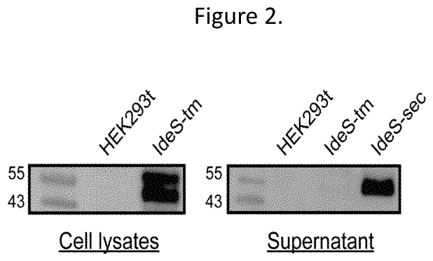

Stable expression of IdeS was tested using immunoblot in a model stable T cell line, such as Jurkat cells. The cell lysates and supernatants of the cell lines were tested using an anti-HA antibody, as a C-terminal HA-tag was included in the IdeS construct. As shown in FIG. 2, expression of IdeS can be adapted successfully to mammalian cells through the transient transfect of HEK293t cells. The function of the CAR T-cells was evaluated with respect to IdeS activity and also CAR activity.

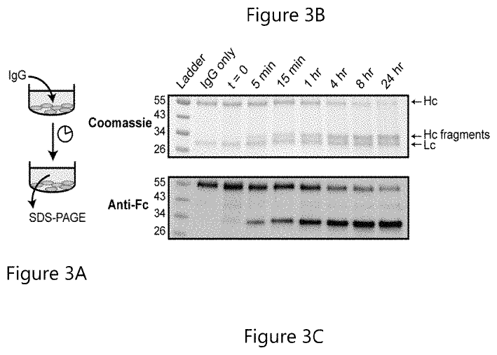

The enzymatic activity of IdeS was tested by evaluating the extent of cleavage of IgG. SDS-PAGE assays as well as an ELISA-based assay were used. FIGS. 3A-3C show the results of the SDS-PAGE assays. For the SDS-PAGE assays, HEK 293t cells were transiently transfected with IdeS-tm. 48 hours post transfection, IgG was added to the wells of a 24-well plate, then removed at different time points and quenched using Laemmli buffer. See FIG. 3A. Cleavage of human polyclonal IgG was tracked over time and detected using an SDS-PAGE assay, which was visualized by both Coomassie and immunoblot (FIG. 3B and 3CC). As shown in FIGS. 3B and 3C, IdeS expressed from HEK293t cells was active in vitro.

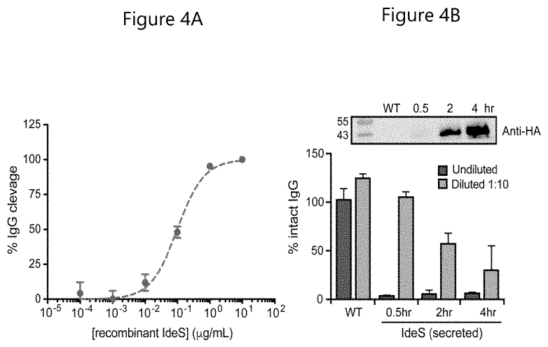

FIGS. 4A and 4B show the results of the ELISA-based assay. An adapted ELISA-based assay (Järnum et al., Molecular Cancer Therapeutics (2017); 16:1887-1897) was used to detect the cleavage of IgG by IdeS. Using recombinant IdeS, the cleavage ELISA was validated (see FIG. 4A). HEK293t cells were transfected with the secreted version of IdeS (“IdeS-sec”). The expression of the enzyme was validated by testing the supernatant fluid by immunoblot using anti-HA (see FIG. 4B). By applying the cleavage ELISA, it was confirmed that IdeS-sec efficiently cleaved human polyclonal IgG at different time points (see FIG. 4B).

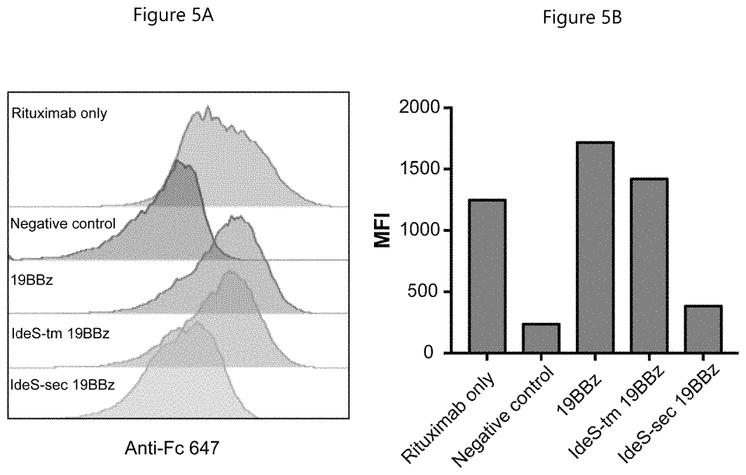

To evaluate whether IdeS can successfully cleave antibodies bound to the surface of a cell, a co-culture experiment was designed. In the co-culture experiment, IdeS-expressing cells were incubated with Raji cells expressing CD20and antibody Rituximab, which bind to the Raji cells. However, IdeS cleaved the antibody releasing the Fc fragments. As shown in FIG. 5, HEK293t cells secreting IdeS successfully cleaved the Fc fragment of Rituximab bound on Raji cells.

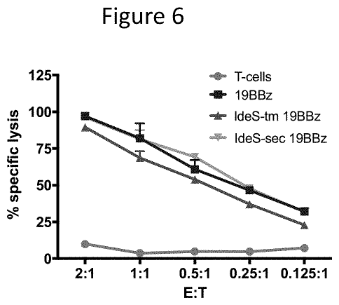

Specific lysis by CAR T-cells was tested by testing the activity against luciferase-expressing target cells as previously disclosed in Dao et al. Science Translational Medicine (2013); 5:1-11). A CD19⁺ Raji cell line was used as a tumor model, and the specific lysis of Raji cells was measured. The Raji cells were modified to express Firefly Luciferase, which allows for a luciferase-based killing assay. As shown in FIG. 6, both IdeS-tm 19BBz cells and IdeS-sec 19BBz cells killed the Raji cells in vitro to the same extent as the 19BB cells without IdeS. Thus, the addition of IdeS to the CAR T-cells does not compromise the killing activity of the CAR T-cells.

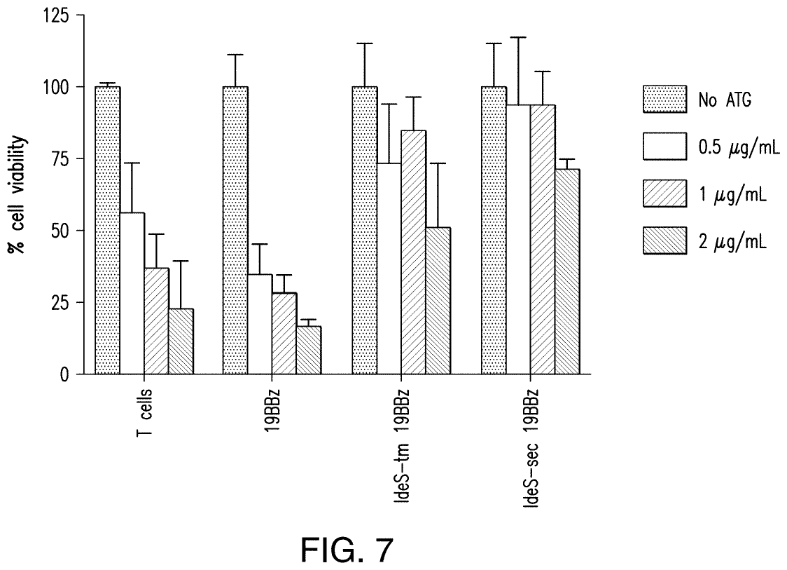

Furthermore, it was studied whether the IdeS-expressing cells can be protected from complement-dependent cytotoxicity (CDC). Untransduced T cells, IdeS-tm 19BBz T-cells, IdeS-sec 19BBz T-cells, andl9BBz T-cells without IdeS were treated with different concentrations of rabbit antithymocyte globulin (ATG) followed by the addition of rabbit serum and incubated for one hour. Cell viability was measured via Cell Titer Glo. As shown in FIG. 7, both IdeS-tm 19BBz T-cells and IdeS-sec 19BBz T-cells cleaved off the Fc fragments of IgG thus evading CDC.

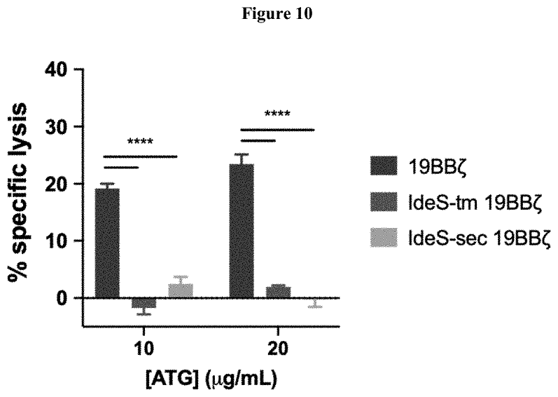

Furthermore, it was studied whether the IdeS-expressing cells can be protected from antibody-dependent cellular cytotoxicity (ADCC). IdeS-tm 19BBz T-cells, IdeS-sec 19BBz T-cells, and 19BBz T-cells without IdeS were treated with different doses of anti-thymocyte globulin (ATG) and subsequently with human PBMCs. Cytotoxicity was determined using a ⁵¹Cr release assay. As shown in FIG. 10, both IdeS-tm 19BBz T-cells and IdeS-sec 19BBz T-cells were protected from lysis compared to the 19BBz T-cells without IdeS.

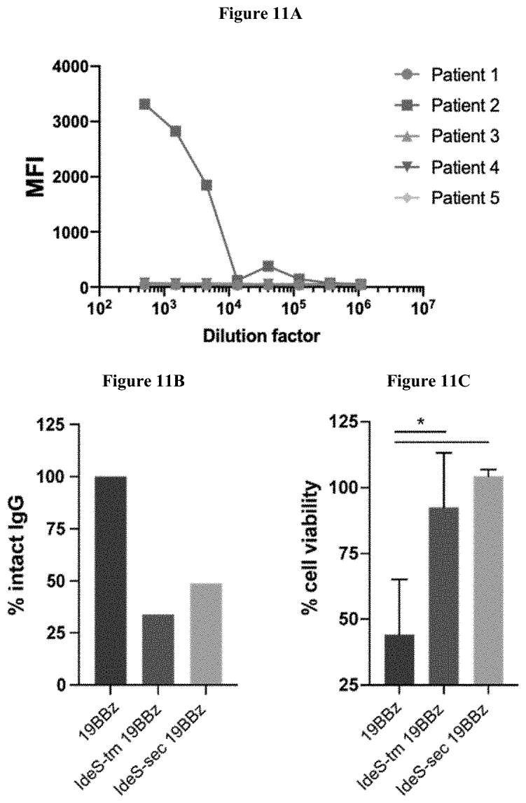

Additionally, the inventors studied whether the IdeS-expressing cells can cleave serum IgG from a kidney transplant patient and be protected from complement-dependent cytotoxicity (CDC). As shown in FIG. 11A, serum derived from a kidney transplant patient (patient 2) containing anti-HLA antibodies causing rejection was shown to bind A02+ cells by flow cytometry. As shown in FIG. 11B, the serum from patient 2 was cleaved by A02+ IdeS-tm 19BBz T-cells and IdeS-sec 19BBz T-cells, as verified by flow cytometry. As shown in FIG. 11C, A02+ IdeS-tm 19BBz T-cells and IdeS-sec 19BBz T-cells were also protected from complement killing (CDC) mediated by patient 2 serum (right).

### Mechanism of Actions

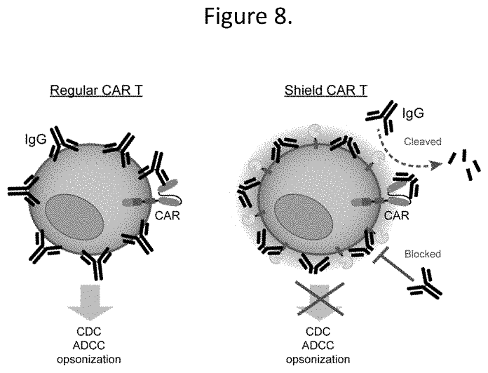

The inventors then studied how the IdeS-expressing cells shield themselves against potential antibodies. FIG. 8 shows one proposed mechanism of action. As shown in FIG. 8, IgG antibodies bind to cell surface antigens and receptors leading to cell death via CDC, ADCC and opsonization. The IdeS cleaves IgG below the hinge region, releasing Fc fragments. The IdeS-expressing cells remain coated in F(ab')2 fragments, which prevents further antibody from binding.

It is important to understand the mechanism of cleavage of the enzyme being secreted versus being membrane bound. The membrane-bound form allows for more localized activity, however, data shown in FIG. 5 suggests the secreted form is more efficient in cleaving antibodies in a trans system. The mechanism of cis or trans cleavage of IdeS-expressing cells is also assessed. For this purpose, a rabbit anti-mouse antibody is used, which antibody targets the murine portion of the scFv. It is evaluated whether the membrane-bound or secreted versions of IdeS are more advantageous.

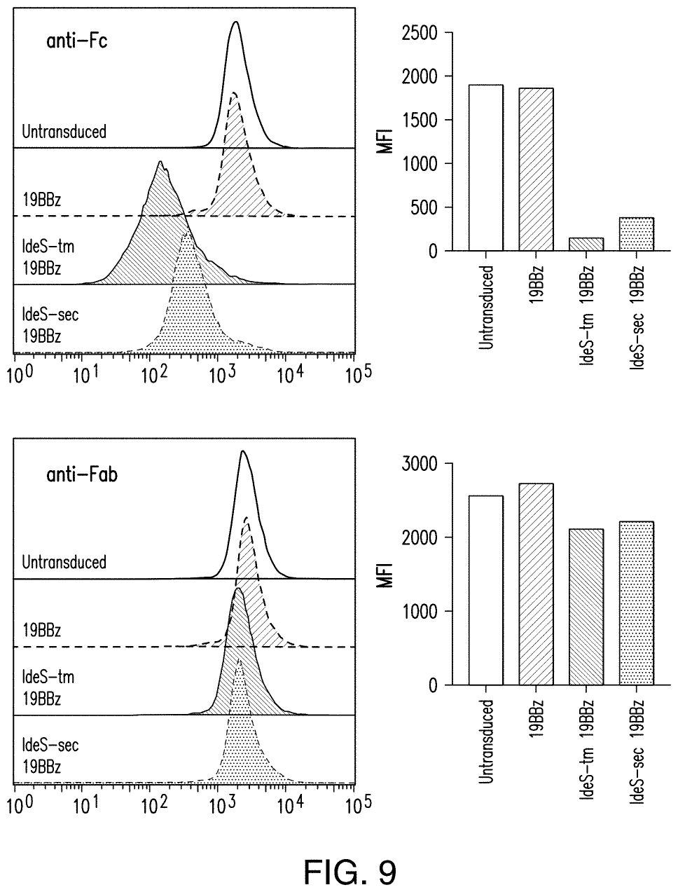

An important aspect of the mechanism of this enzyme, is that since it cleaves IgG below the hinge region, the F(ab′)₂ fragments remain intact, and thus, can remain bound on the surface of cells. It was observed that using recombinant IdeS that with increasing concentrations of IdeS, the Fc fragments were removed from the surface of cells, while the F(ab′)₂ fragments remained bound to the surface. As shown in FIG. 9, CAR T cells expressing IdeS cleaved IgG Fc and maintained F(ab′)₂ shield. After IgG (ATG) was added to the IdeS expressing cells (either the transmembrane or secreted forms), the IgG stayed bound to the cell as an Fab form and the Fc was gone. The lack of the Fc eliminated the function while the bound Fab prevented new cytotoxic IgG from binding, hence, functioned as a shield. With cells that contained no IdeS, this did not happen.

This suggests that the residual F(ab')2 fragments creates a shield around the cell, hiding it from a potential future humoral response. This hypothesis is tested by using membrane-bound and cell-secreted IdeS in the stable lines described above. Using an anti-Fab specific antibody or an anti-Fc specific antibody, it is detected what portion of IgG is still bound to a cell. The kinetics of this process is also examined, to understand how long after cleavage the F(ab′)₂ fragments remain bound, and whether they are competed off by intact IgG.

### Conclusions

Our data shown in this Example demonstrate that the IdeS-expressing CAR-T cells were successfully expressed in mammalian cells, and showed efficient in vitro cleavage activity. In addition, the inclusion of IdeS to the CAR T-cells does not compromise the in vitro killing activity of the CAR. Furthermore, the IdeS-expressing CAR-T cells were protected against complement-dependent cytotoxicity (CDC).

### Example 2

### In Vivo Activities

### Summary

In this Example, the in vivo activities of the IdeS-expressing CAR T-cells of Example 1 are investigated. The IgG cleavage activity and cell killing activity of these cells are investigated in an in vivo mouse model. These cells are tested in a system where they are targeted by an antibody to reproduce a model of humoral immunogenicity to CAR T-cells. With this model, the persistence of the cells is tested as well as killing efficiency of tumor cells over regular CAR T-cells. One important consideration in the design of these experiments is that IdeS does not cleave mouse IgG; therefore. Thus, NSG mice, which is a highly immunodeficient mouse model compatible with added xenogeneic antibodies (such as rabbit or human, on which IdeS acts, as well as potentially engrafted immune effector cells), are used.

### In Vivo Cell Persistence

The in vivo persistence of the IdeS-expressing CAR T-cells is compared to CAR T-cells without IdeS is tested. NSG mice are injected intraperitoneally (IP) with the IdeS-expressing CAR T-cells or CAR T-cells without IdeS. The mice are then be injected IP with a human antibody targeting the CAR T-cells, such as anti-CD3, anti-MHC class I antibody, or an antibody targeting the CAR. Rabbit or human derived antibodies are used, as IdeS fully cleaves all isoforms of rabbit IgG as well as human IgG (Johansson et al., PLoS ONE (2008); 3:1-6; Yang et al., Nephrology Dialysis Transplantation (2010); 25:2479-2486; Wang et al., Experimental Neurology (2017); 291:134-140). The IdeS-expressing cells are expected to cleave the antibodies below the hinge and thus release the Fc fragments. Ascites and peripheral blood harvested from the mice are analyzed at different time points by immunoblot or ELISA, as previously shown (Rafiq et al., Nature Biotechnology (2018); 36:847-858), to evaluate cleavage of IgG (as shown in FIGS. 3 and 4). The presence of CAR T-cells is determined by collecting ascites, and analyzing by flow cytometry with anti-CD19 CAR and anti-HA tag, which is present on the IdeS construct. To investigate whether Fab fragments remain bound to the cell surface (as a shield against further binding of functional IgG), anti-Fab specific antibodies are used to analyze residual binding by flow cytometry. The presence of remaining available CAR T-cell surface target (i.e. CD3 or MHC I) is also assessed. Similar experiments re conducted with intravenous (IV) infusions of cells and antibodies.

### In Vivo Efficacy

A CD19⁺ Raji cell line is used as a tumor model, and the specific lysis of Raji cells is measured. The Raji cells are modified to express Firefly Luciferase, which allows for a luciferase-based killing assay as well as in vivo bioluminescence imaging of the tumor (Koneru et al., Oncoimmunology (2015); 4:e994446). NSG mice are infused IP with Raji cells, followed by the IdeS-expressing CAR-T cells or CAR T-cells without IdeS, and injection of a CAR T-cell targeting antibody that does not bind to the Raji cells (e.g., anti-CD3 or an anti-mouse antibody) to target the CAR. Tumor growth and disease progression are evaluated using bioluminescence imaging. Persistence of the CAR T-cells are also be evaluated in this system using flow cytometry or by imaging of the CAR T cells carrying an alternative luminescent probe. CAR T-cells engineered to express Gaussia luciferase can be used orthogonally to Firefly luciferase (Santos et al., Nature Medicine (2009); 15:338-344). CART cells carrying an luminescent probe allow to track and monitor CAR T-cell persistence over time, while also tracking the tumor. Similar experiments are conducted with IV infusions of cells and antibodies.

### Example 3

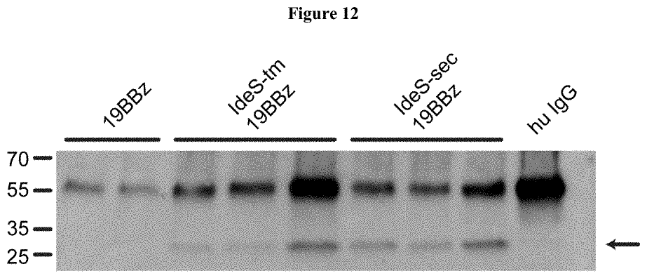

Human T cells were transduced with the 19BBz without IdeS CAR (Lanes from left: #1-2) or IdeS-tm 19BBz CAR (transmembrane form) (Lanes from left: #3-5) and IdeS-sec 19BBz CAR (secreted form) (Lanes from left: #6-8). 2×10⁶ CAR T cells were injected i.p. in NSG mice and after 24 hour human polyclonal IgG was also injected i.p. Cleavage of IgG was assessed by performing an i.p. lavage using PBS, purifying the samples using magnetic protein G beads, and analyzing by Western Blot using an anti-human Fc-specific HRP secondary antibody. As shown in FIG. 12, un-cleaved heavy chain was observed around 55 kDa (lane #9), while cleaved Fc fragments were present around 25 kDa (arrow).

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof

All patents and publications mentioned in this specification are herein incorporated by reference to the same extent as if each independent patent and publication was specifically and individually indicated to be incorporated by reference.

What is claimed is:

1. A cell comprising:
(a) a ligand-recognizing receptor, and
(b) an IgG-degrading enzyme or a fragment thereof.

2. The cell of claim 1, wherein the IgG-degrading enzyme is secreted.

3. The cell of claim 1, wherein the IgG-degrading enzyme is membrane bound.

4. The cell of claim 3, further comprising (c) a transmembrane domain attached to the IgG-degrading enzyme, optionally wherein the transmembrane domain is attached to the C-terminus of the IgG-degrading enzyme, and/or the transmembrane domain attached to the IgG-degrading enzyme comprises a CD8 polypeptide, optionally wherein the CD8 polypeptide comprises a transmembrane of CD8.

5. The cell of claim 1, wherein the IgG-degrading enzyme is selected from IgG-degrading enzyme of S. pyogenes (IdeS), IgG-degrading enzyme of S. equi subsp. zooepidemicus (IdeZ), IgG-degrading enzyme of S. equi subsp. equi. (IdeE), an endoglycosidase from Streptococcus pyogenes (EndoS), and streptococcal cysteine proteinase from Streptococcus pyogenes (SpeB).

6. The cell of claim 1, wherein the ligand-recognizing receptor is recombinantly expressed and/or expressed from a vector; and/or the IgG-degrading enzyme is expressed from a vector.

7. The cell of claim 1, wherein the cell is a responsive cell or an activatable cell, optionally wherein the cell is an immunoresponsive cell.

8. The cell of claim 1, wherein the cell is selected from the group consisting of T cells, Natural Killer (NK) cells, B cells, macrophages, monocytes, dendritic cells, stem cells, and normal tissue cells, optionally wherein the cell a T cell.

9. The cell of claim 1, wherein the ligand-recognizing receptor binds to an antigen, optionally wherein the antigen is selected from the group consisting of a tumor antigen, a pathogen antigen, a normal cell antigen, an HLA antigen, and an alloantigen.

10. The cell of claim 9, wherein the antigen is a tumor antigen, optionally wherein the tumor antigen is CD19.

11. The cell of claim 9, wherein the antigen is an HLA antigen or an alloantigen, optionally wherein the alloantigen is a minor histocompatibility alloantigen.

12. The cell of claim 1, wherein the ligand-recognizing receptor is a T cell receptor (TCR) or a chimeric antigen receptor (CAR), optionally wherein the ligand -recognizing receptor is a CAR.

13. The cell of claim 12, wherein the CAR comprises an extracellular antigen-binding domain, a transmembrane domain, and an intracellular signaling domain, optionally wherein
a) the extracellular antigen-binding domain of the CAR comprises a single chain variable fragment (scFv);
b) the transmembrane domain comprises a CD8 polypeptide; and/or
c) the intracellular signaling domain of the CAR comprises a CD3ζ polypeptide, optionally wherein the intracellular signaling domain of the CAR further comprises at least one co-stimulatory signaling domain.

14. The cell of claim 13, wherein the at least one co-stimulatory domain comprises a CD28 polypeptide, a 4-1BB polypeptide, or a combination thereof, optionally wherein the at least one co-stimulatory domain comprises a 4-1BB polypeptide.

15. The cell of claim 1, wherein the IgG-degrading enzyme (a) cleaves an IgG, thereby preventing an IgG antibody from killing the cell, and/or (b) cleaves an IgG, thereby allowing the remaining fragment of the IgG to retain the binding to the cell, which protects the cells from one or more cytotoxic antibodies, optionally the one or more cytotoxic antibodies bind to the same epitope region as the IgG and kill the cell.

16. A composition comprising a cell of claim 1, optionally wherein the composition is a pharmaceutical composition further comprising a pharmaceutically acceptable excipient.

17. A method for producing a cell, the method comprising introducing into a cell (a) a first polynucleotide encoding a ligand-recognizing receptor; and (b) a second polynucleotide encoding an IgG-degrading enzyme or a fragment thereof, wherein each of the first and second nucleic acid sequence optionally operably linked to a promoter element.

18. A nucleic acid composition comprising (a) a first polynucleotide encoding a ligand-recognizing receptor and (b) a second polynucleotide encoding an IgG-degrading enzyme or a fragment thereof.

19. A vector comprising the nucleic acid composition of claim 18.

20. A kit comprising a cell of claim 1.

21. A method of reducing tumor burden in a subject, the method comprising administering to the subject the cell of claim 1.

22. A method of treating and/or preventing a neoplasia, a pathogen infection, and/or an autoimmune disease, the method comprising administering to the subject the cell of claim 1.

23. A method of lengthening survival of a subject having a neoplasia, a pathogen infection, and/or an autoimmune disease, the method comprising administering to the subject the cell of claim 1.

24. A method of reducing and/or preventing an antibody-mediated rejection of cells and/or tissues in a subject who receives an organ transplant, comprising administering the cell of claim 1.

25. A method of reducing and/or preventing an antibody-mediated rejection of cells or tissues that are used in a subject who receives a cell therapy, comprising administering the cell of claim 1.


SEQUENCE LISTING (45 sequences)

SEQ ID NO: 1 - protein, 341 aa, Artificial Sequence
  Synthetic IdeS amino acid sequence
MRKRCYSTSAVVLAAVTLFALSVDRGVIADSFSANQEIRYSEVTPYHVTSVWTKGVTPPAKFTQGEDVFHAPYVANQGWYDITKTFNGKDDLLCGAATAGNMLHWWFDQNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 2 - DNA, 936 bp, Artificial Sequence
  Synthetic exemplary nucleic acid sequence encoding amino acids 30 to 341 of SEQ ID NO 1
gactcttttagtgccaatcaagaaatccgatatagcgaggtgactccttaccatgtaacttctgtgtggaccaagggagttaccccaccagccaagttcacgcagggtgaggacgtatttcacgcaccgtacgtagctaaccagggttggtacgacatcactaagaccttcaatgggaaagacgatcttttgtgtggtgccgcaacggcgggcaacatgctgcactggtggttcgaccaaaacaaggagaagatcgaagcgtacttgaagaaacacccagacaaacagaaaatcatgtttggagaccaggagctcctggatgtgagaaaggtaatcaacactaaaggtgaccaaacaaacagtgaactttttaactattttcgggacaaggcgtttccaggattgagtgccagaagaatcggcgtaatgcctgacctcgtgcttgacatgttcatcaatggatactatctcaatgtatataagacccaaaccacagatgttaatcgaacttatcaggagaaggatagaaggggaggaatatttgatgccgtttttactcgaggagaccagtctaagctcttgaccagcaggcacgacttcaaagagaagaatcttaaagaaatatctgatctcataaagaaggaactgacggaaggcaaagcgctgggactttcccatacgtatgccaatgtaagaatcaatcatgtcataaacctttggggtgctgatttcgattctaatggaaatcttaaggctatatatgttactgattccgactccaacgcgtctattggcatgaaaaaatacttcgtcggggtgaactcagcaggaaaggtcgcaatatctgctaaggaaattaaggaagacaacataggggcgcaagtgctgggtctcttcaccctttccaccggccaagactcctggaatcaaacaaat

SEQ ID NO: 3 - protein, 71 aa, Artificial Sequence
  Synthetic CD8 polypeptide
PTTTPAPRPPTPAPTIASQPLSLRPEACRPAAGGAVHTRGLDFACDIYIWAPLAGTCGVLLLSLVITLYCN

SEQ ID NO: 4 - DNA, 213 bp, Artificial Sequence
  Synthetic exemplary nucleic acid sequence encoding the amino acid of SEQ ID NO 3
ccaactactactcccgcaccgagaccgcccactcctgctcccacgattgcctcccaacctcttagcttgagaccggaagcatgtcggcctgcggccggtggcgcagtacatactcgcggcctggactttgcgtgcgacatatacatttgggcacccctggccggcacttgcggagttttgctgctgtctctcgtgataactctctattgtaac

SEQ ID NO: 5 - protein, 20 aa, Homo sapiens
  misc_feature (1)..(20): human IL-2 signal peptide
MYRMQLLSCIALSLALVTNS

SEQ ID NO: 6 - protein, 20 aa, Mus musculus
  misc_feature (1)..(20): mouse IL-2 signal peptide
MYSMQLASCVTLTLVLLVNS

SEQ ID NO: 7 - protein, 20 aa, Homo sapiens
  misc_feature (1)..(20): human kappa signal sequence
METPAQLLFLLLLWLPDTTG

SEQ ID NO: 8 - protein, 20 aa, Mus musculus
  misc_feature (1)..(20): mouse kappa signal sequence
METDTLLLWVLLLWVPGSTG

SEQ ID NO: 9 - protein, 21 aa, Homo sapiens
  misc_feature (1)..(21): human CD8 signal peptide
MALPVTALLLPLALLLHAARP

SEQ ID NO: 10 - protein, 18 aa, Homo sapiens
  misc_feature (1)..(18): truncated human CD8 signal peptide
MALPVTALLLPLALLLHA

SEQ ID NO: 11 - protein, 16 aa, Homo sapiens
  misc_feature (1)..(16): human albumin signal sequence
MKWVTFISLLFSSAYS

SEQ ID NO: 12 - protein, 30 aa, Homo sapiens
  misc_feature (1)..(30): human prolactin signal sequence
MDSKGSSQKGSRLLLLLVVSNLLLCQGVVS

SEQ ID NO: 13 - DNA, 54 bp, Artificial Sequence
  Synthetic CD8 signal sequence nucleic acid sequence encoding the amino acid sequence of SEQ ID NO 10
atggcccttccggtgacggcgcttctcctccctttggcgcttcttctgcacgct

SEQ ID NO: 14 - protein, 269 aa, Mus musculus
  misc_feature (1)..(269): murine CD19 polypeptide
RPQKSLLVEVEEGGNVVLPCLPDSSPVSSEKLAWYRGNQSTPFLELSPGSPGLGLHVGSLGILLVIVNVSDHMGGFYLCQKRPPFKDIWQPAWTVNVEDSGEMFRWNASDVRDLDCDLRNRSSGSHRSTSGSQLYVWAKDHPKVWGTKPVCAPRGSSLNQSLINQDLTVAPGSTLWLSCGVPPVPVAKGSISWTHVHPRRPNVSLLSLSLGGEHPVREMWVWGSLLLLPQATALDEGTYYCLRGNLTIERHVKVIARSAVWLWLLRTGG

SEQ ID NO: 15 - protein, 272 aa, Homo sapiens
  misc_feature (1)..(272): human CD19 polypeptide
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 16 - protein, 122 aa, Artificial Sequence
  Synthetic VH extracellular antigen-binding domain of CAR
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 17 - protein, 108 aa, Artificial Sequence
  Synthetic VL extracellular antigen-binding domain of CAR
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 18 - protein, 15 aa, Artificial Sequence
  Synthetic linker
GGGGSGGGGSGGGGS

SEQ ID NO: 19 - protein, 7 aa, Artificial Sequence
  Synthetic VH CDR1
GYAFSSY

SEQ ID NO: 20 - protein, 6 aa, Artificial Sequence
  Synthetic VH CDR2
YPGDGD

SEQ ID NO: 21 - protein, 13 aa, Artificial Sequence
  Synthetic VH CDR3
KTISSVVDFYFDY

SEQ ID NO: 22 - protein, 11 aa, Artificial Sequence
  Synthetic VL CDR1
KASQNVGTNVA

SEQ ID NO: 23 - protein, 7 aa, Artificial Sequence
  Synthetic VL CDR2
SATYRNS

SEQ ID NO: 24 - protein, 9 aa, Artificial Sequence
  Synthetic VL CDR3
QQYNRYPYT

SEQ ID NO: 25 - protein, 245 aa, Artificial Sequence
  Synthetic scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 26 - DNA, 735 bp, Artificial Sequence
  Synthetic nucleotide sequence encoding the amino acid sequence of SEQ ID NO 25
gaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgg

SEQ ID NO: 27 - protein, 235 aa, Artificial Sequence
  Synthetic CD8 polypeptide
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 28 - DNA, 213 bp, Artificial Sequence
  Synthetic exemplary nucleotide sequence encoding amino acids 137 to 207 of SEQ ID NO 27
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 29 - protein, 247 aa, Mus musculus
  misc_feature: CD8 polypeptide
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 30 - protein, 220 aa, Homo sapiens
  misc_feature: CD28 polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 31 - protein, 164 aa, Homo sapiens
  misc_feature: CD3-zeta polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 32 - protein, 188 aa, Mus musculus
  misc_feature: CD3-zeta polypeptide
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQRRRNPQEGVYNALQKDKMAEAYSEIGTKGERRRGKGHDGLYQDSHFQAVQFGNRREREGSELTRTLGLRARPKACRHKKPLSLPAAVS

SEQ ID NO: 33 - protein, 58 aa, Artificial Sequence
  Synthetic CD3-zeta polypeptide
RVKFSRSADAPAYQQGQNQLYNELNLGRREEYDVLDKRRGRDPEMGGKPRRKNPQEGL

SEQ ID NO: 34 - DNA, 174 bp, Artificial Sequence
  Synthetic exemplary nucleic acid sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctg

SEQ ID NO: 35 - protein, 255 aa, Homo sapiens
  misc_feature: 4-1BB polypeptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 36 - DNA, 126 bp, Artificial Sequence
  Synthetic exemplary nucleic acid sequence encoding amino acids 214 to 255 of SEQ ID NO 35
aaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 37 - protein, 277 aa, Homo sapiens
  misc_feature (1)..(277): human OX40 has the amino acid sequence
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 38 - protein, 199 aa, Homo sapiens
  misc_feature: human ICOS
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 39 - protein, 473 aa, Artificial Sequence
  Synthetic 19BBz
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 1419 bp, Artificial Sequence
  Synthetic exemplary nucleic acid sequence encoding the amino acid sequence of SEQ ID NO 39
gaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 41 - DNA, 54 bp, Artificial Sequence
  Synthetic exemplary nucleic acid sequence encoding the amino acid sequence of SEQ ID NO 10
atggctctcccagtgactgccctactgcttcccctagcgcttctcctgcatgca

SEQ ID NO: 42 - protein, 491 aa, Artificial Sequence
  Synthetic 19BBz comprising the CD8 signal peptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - DNA, 1473 bp, Artificial Sequence
  Synthetic exemplary nucleic acid sequence encoding the amino acid sequence of SEQ ID NO 42
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 44 - protein, 19 aa, Artificial Sequence
  Synthetic P2A peptide
ATNFSLLKQAGDVEENPGP

SEQ ID NO: 45 - DNA, 57 bp, Artificial Sequence
  Synthetic exemplary nucleotide sequence encoding the amino acid sequence set forth in SEQ ID NO 44
gctactaacttcagcctgctgaagcaggctggagacgtggaggagaaccctggacct